(19) United States
(12) Patent Application Publication
(10) Pub. No.: US 2008/0009531 A1
(43) Pub. Date: Jan. 10, 2008

(54) 5-Anilino-4-Heteroarylpyrazole Derivatives Useful for the Treatment of Diabetes

(75) Inventors: Louis David Cantin, Hamden, CT (US); Xin Ma, Bethany, CT (US); Christiana Akuche, Hamden, CT (US); Sidney X. Liang, Bethany, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporation, West Haven, CT (US)

(21) Appl. No.: 11/596,959

(22) PCT Filed: May 20, 2005
(86) PCT No.: PCT/US05/17889

§ 371 Date: Nov. 17, 2006

Related U.S. Application Data
(60) Provisional application No. 60/573,066, filed on May 20, 2004.

(51) Int. Cl. A61K 31/4155; A61K 31/4164; A61K 31/422; A61K 31/427; A61P 3/04; A61P 3/10; A61P 9/00; C07D 231/38; C07D 403/04; C07D 405/04; C07D 409/04; C07D 413/04; C07D 417/04; C07D 493/00; C07D 495/00

(52) U.S. Cl. 514/369; 514/374; 514/378; 514/397; 514/407; 548/187; 548/236; 548/247; 548/312.4; 548/365.7; 548/371.7

(57) ABSTRACT

The present invention relates to 5-anilino-4-heteroarylpyrazole compounds, pharmaceutical compositions, and methods for treating diabetes and related disorders.

This application claims benefit of U.S. Provisional Application Ser. No. 60/573,066; filed on May 20, 2004, the contents of which are incorporated herein by reference in their entirety.

### FIELD OF THE INVENTION

The present invention relates to 5-anilino-4-heteroarylpyrazole compounds, pharmaceutical compositions, and methods for treating diabetes and related disorders.

### BACKGROUND OF THE INVENTION

Diabetes is characterized by impaired glucose metabolism manifesting itself among other things by an elevated blood glucose level in the diabetic patient. Underlying defects lead to a classification of diabetes into two major groups. Type 1 diabetes, or insulin dependent diabetes mellitus (IDDM), arises when patients lack insulin-producing beta-cells in their pancreatic glands. Type 2 diabetes, or non-insulin dependent diabetes mellitus (NIDDM), occurs in patients with impaired beta-cell function and alterations in insulin action.

The current treatment for type 1 diabetic patients is injection of insulin, while the majority of type 2 diabetic patients are treated with agents that stimulate beta-cell function or with agents that enhance the tissue sensitivity of the patients towards insulin. The drugs presently used to treat type 2 diabetes include alpha-glucosidase inhibitors, insulin sensitizers, insulin secretagogues, and metformin.

Over time, almost one-half of type 2 diabetic subjects lose their response to these agents. Insulin treatment is instituted after diet, exercise, and oral medications have failed to adequately control blood glucose. The drawbacks of insulin treatment are the need for drug injection, the potential for hypoglycemia, and weight gain.

Because of the problems with current treatments, new therapies to treat type 2 diabetes are needed. In particular, new treatments to retain normal (glucose-dependent) insulin secretion are needed. Such new drugs should have the following characteristics: dependency on glucose for promoting insulin secretion (i.e., compounds that stimulate insulin secretion only in the presence of elevated blood glucose); low primary and secondary failure rates; and preservation of islet cell function.

### DETAILED DESCRIPTION OF THE INVENTION

The invention provides anilinopyrazole derivatives of Formula (I) 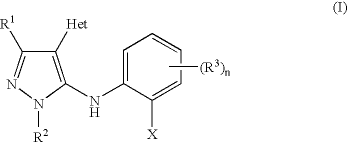
wherein
R¹ is H,
(C₁-C₆)alkyl optionally substituted with one substituent selected from the group consisting of (C₁-C₄)alkoxy, phenyl optionally substituted with halo, and [tri(C₁-C₄)alkyl]silyl,
(C₃-C₆)alkenyl,
(C₃-C₆)alkynyl,
(C₃-C₆)cycloalkyl optionally substituted with up to two substituents selected from the group consisting of (C₁-C₃)alkyl, CF₃, and halo,
(C₁-C₆)haloalkyl, or
phenyl optionally substituted with up to four substituents selected from the group consisting of
   halo,
   (C₁-C₆)alkyl optionally substituted with one (C₁-C₄)alkoxy or oxo,
   (C₁-C₆)alkoxy,
   (C₁-C₃)haloalkyl,
   (C₁-C₃)haloalkoxy,
   (C₃-C₆)cycloalkyl,
   NR⁴R⁴,
   cyano, and
   (C₁-C₆)alkylthio;
      Het is a mono heterocyclic ring radical selected from the group consisting of thienyl, furyl, oxazolyl, isoxazolyl, imidazolyl, thiazolyl, isothiazolyl, pyrrolyl, pyrazolyl, and thiadiazolyl,
each of which may be optionally substituted with up to two substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo,
or
optionally fused to a 5- or 6-membered saturated or partially saturated carbocyclic ring or to a 5- or 6-membered saturated or unsaturated heterocyclic ring containing 1-3 heteroatoms selected from N, O, and S,
or
is a bicyclic heterocylic ring radical selected from the group consisting of 2-benzothienyl, 3-benzothienyl, 2-benzofuryl, 3-benzofuryl, 2-benzoazolyl, and 2-benzothiazolyl
   each of which may be optionally substituted with up to four substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo;
      R² is (C₁-C₆)alkyl,
(C₃-C₆)cycloalkyl,
(C₂-C₃)haloalkyl,
benzyl optionally substituted on the aryl ring with up to four substituents selected from the group consisting of
   (C₁-C₆)alkyl optionally substituted with one (C₁-C₄)alkoxy or oxo,
   halo,
   (C₁-C₃)haloalkyl,
   (C₁-C₆)alkoxy,
   (C₁-C₃)haloalkoxy,
   NR⁴R⁴,
   cyano,
   (C₁-C₆)alkylthio, and
   SO₂(C₁-C₃)alkyl,
or
phenyl optionally substituted with up to four substituents selected from the group consisting of
   (C₁-C₆)alkyl optionally substituted with one (C₁-C₄)alkoxy or oxo,
   halo,
   (C₁-C₃)haloalkyl,
   (C₁-C₆)alkoxy,
   (C₁-C₃)haloalkoxy,
   NR⁴R⁴,
   cyano,
   (C₁-C₆)alkylthio, and
   SO₂(C₁-C₃)alkyl;
      R³ is (C₁-C₆)alkyl optionally substituted with one (C₁-C₄)alkoxy or oxo,
(C₁-C₆)alkoxy,
(C₁-C₆)alkylthio,
(C₁-C₃)haloalkyl,
(C₁-C₃)haloalkoxy,
halo, or
NR⁴R⁴;
   n=0, 1, 2, or 3;
   X is CO₂R⁴;
   R⁴ is H,
(C₁-C₆)alkyl,
benzyl optionally substituted on the aryl ring with up to four substituents selected from the group consisting of
   halo,
   (C₁-C₆)alkyl optionally substituted with one (C₁-C₄)alkoxy,
   (C₁-C₃)alkoxy,
   (C₁-C₃)haloalkyl,
   (C₁-C₃)haloalkoxy,
   cyano, and
   (C₁-C₆)alkylthio,
or
phenyl optionally substituted with up to four substituents selected from the group consisting of
   (C₁-C₆)alkyl optionally substituted with one (C₁-C₄)alkoxy,
   halo,
   (C₁-C₆)alkoxy,
   (C₁-C₃)haloalkyl,
   (C₁-C₃)haloalkoxy,
   cyano, and
   (C₁-C₆)alkylthio;
or the pharmaceutically acceptable salts thereof.

The terms identified above have the following meaning throughout:

The term “halo” means F, Br, Cl, and I.

The terms “(C₁-C₃)alkyl” and “(C₁-C₆)alkyl” mean a linear or branched saturated hydrocarbon radical having from about 1 to about 3 C atoms or about 1 to about 6 C atoms, respectively. Such groups include, but are not limited to, methyl, ethyl, n-propyl, isopropyl, butyl, isobutyl, pentyl, hexyl, and the like.

The term “(C₃-C₆)alkenyl” means a linear or branched unsaturated hydrocarbon radical containing a double bond and from about 3 to about 6 carbon atoms. The double bond may be between any two available carbon atoms in the chain. Such groups include, but are not limited to, allyl, isopropenyl, 2-butenyl, 2-ethyl-2-butenyl, 1-hexenyl, and the like.

The term “(C₃-C₆)alkynyl” means a linear or branched unsaturated hydrocarbon radical containing a triple bond and from about 3 to about 6 carbon atoms. The triple bond may be between any two available carbon atoms in the chain. Such groups include, but are not limited to, propargyl, 2-butynyl, 1-methyl-2-butynyl, 3-hexynyl, and the like.

The term “(C₃-C₆)cycloalkyl” includes, but are not limited to, cyclopropyl, cyclobutyl, cyclopentyl, and cyclohexyl.

The terms “(C₁-C₃)alkoxy,” “(C₁-C₄)alkoxy,” and “(C₁-C₆)alkoxy” mean a linear or branched saturated hydrocarbon radical having from about 1 to about 3 C atoms, about 1 to about 4 C atoms, or about 1 to about 6 C atoms, respectively, said radical being attached to an O atom. The O atom is the atom through which the alkoxy substituent is attached to the rest of the molecule. Such groups include, but are not limited to, methoxy, ethoxy, n-propoxy, isopropoxy, butoxy, pentyloxy, hexyloxy, and the like.

The term “(C₁-C₃)haloalkoxy” means a (C₁-C₃)alkoxy group, substituted on C with a halogen atom. Such groups include, but are not limited to, trifluoromethoxy, difluoromethoxy, 2,2-difluoroethoxy, 2,2,2-trifluoroethoxy, 2-chloroethoxy, 3-chloropropoxy, 1-fluoro-2,2,-dichloroethoxy, and the like.

The terms “(C₁-C₃)haloalkyl,” “(C₂-C₃)haloalkyl,” and “(C₁-C₆)haloalkyl” mean a (C₁-C₃)alkyl group, (C₂-C₃)alkyl group, or (C₁-C₆)alkyl group substituted on C with a halogen atom. Such groups include, but are not limited to, trifluoromethyl, difluoroethyl, 1-fluoro-2,2-dichloroethyl, 3-chloropropyl, 4-bromohexyl, and the like.

The term “[tri(C₁-C₄)alkylsilyl]” means a Si radical bearing three (C₁-C₄)alkyl substituents, each substituent being independently selected. The Si atom is the atom through which the radical is attached to the rest of the molecule. Such groups include, but are not limited to, trimethylsilyl, tert-butyl-dimethylsilyl, and the like.

The formula “NR⁴R⁴” means that each of the two possible R⁴ groups attached to the N atom are selected independently from the other so that they may be the same or they may be different.

The term “(C₁-C₆)alkylthio” means a linear or branched saturated hydrocarbon radical having from about 1 to about 6 C atoms, respectively, said radical being attached to an S atom. The S atom is the atom through which the alkylthio substituent is attached to the rest of the molecule. Such groups include, but are not limited to, methylthio, ethylthio, n-propylthio, isopropylthio, and the like.

The term “SO₂(C₁-C₃)alkyl” means a linear or branched saturated hydrocarbon radical having from about 1 to about 3 C atoms, said radical being attached to the S atom of the SO₂ group. The S atom of the SO₂ group is the atom through which the SO₂(C₁-C₃)alkyl substituent is attached to the rest of the molecule. Such groups include, but are not limited to, methylsulfonyl, ethylsulfonyl, n-propylsulfonyl and isopropylsulfonyl, and the like.

The term “mono or bicyclic heteroaromatic ring radical” means a 5-membered monocyclic heteroaromatic ring, or a bicyclic ring in which a 5-membered heteroaromatic ring is fused to a 6-membered heteroaromatic or phenyl ring. The connecting bond from the ring is attached to any available position of the 5-membered heteroaromatic ring.

The term “optionally substituted” means that the moiety so modified may have from none to up to at least the highest number of substituents indicated. Each substituent may replace any H atom on the moiety so modified as long as the replacement is chemically possible and chemically stable. When there are two or more substituents on any moiety, each substituent is chosen independently of any other substituent and can, accordingly, be the same or different.

Alternative Forms of Novel Compounds

Also included in the compounds of the present invention are (a) the stereoisomers thereof, (b) the pharmaceutically-acceptable salts thereof, (c) the tautomers thereof, (d) the protected acids and the conjugate acids thereof, and (e) the prodrugs thereof.

The stereoisomers of these compounds may include, but are not limited to, enantiomers, diastereomers, racemic mixtures, and combinations thereof. Such stereoisomers may be prepared and separated using conventional techniques, either by reacting enantiomeric starting materials, or by separating isomers of compounds of the present invention. Isomers may include geometric isomers. Examples of geometric isomers include, but are not limited to, cis isomers or trans isomers across a double bond. Other isomers are contemplated among the compounds of the present invention. The isomers may be used either in pure form or in admixture with other isomers of the inhibitors described above.

Pharmaceutically-acceptable salts of the compounds of the present invention include salts commonly used to form alkali metal salts or form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, heterocyclic, carboxylic, and sulfonic classes of organic acids. Examples of organic and sulfonic classes of organic acids includes, but are not limited to, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, N-hydroxybutyric, salicylic, galactaric, and galacturonic acid, and combinations thereof.

Tautomers of the compounds of the invention are encompassed by the present invention. Thus, for example, a carbonyl includes its hydroxy tautomer.

The protected acids include, but are not limited to, esters, hydroxyamino derivatives, amides and sulfonamides.

The present invention includes the prodrugs and salts of the prodrugs. Formation of prodrugs is well known in the art in order to enhance the properties of the parent compound; such properties include solubility, absorption, biostability, and release time (see, e.g., “Pharmaceutical Dosage Form and Drug Delivery Systems” (Sixth Edition), edited by Ansel et al., publ. by Williams & Wilkins, pgs. 27-29, (1995), which is hereby incorporated by reference). Commonly used prodrugs are designed to take advantage of the major drug biotransformation reactions, and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation, and acetylation (see, e.g., Goodman and Gilman's The Pharmacological Basis of Therapeutics (Ninth Edition), editor Molinoff et al., publ. by McGraw-Hill, pages 11-13, (1996), which is hereby incorporated by reference).

A comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

For purposes of this invention, the chemical elements are identified in accordance with the Periodic Table of the Elements, CAS version, Handbook of Chemistry and Physics, 67th Ed., 1986-87.

General Preparative Methods

In general, the compounds used in this invention may be prepared by standard techniques known in the art, by known processes analogous thereto, and/or by the processes described herein, using starting materials which are either commercially available or producible according to routine, conventional chemical methods. Furthermore, preparative methods described in U.S. patent application Ser. No. 10/719,485; filed Nov. 21, 2003, are incorporated herein by reference. The following preparative methods are presented to aid the reader in the synthesis of the compounds of the present invention.

For example, as illustrated in Reaction Scheme 1, an aminopyrazole of Formula (II) is coupled to a substituted aniline of Formula (III) under Ullman or Buchwald conditions as described in U.S. patent application Ser. No. 10/719,485, to provide the anilinopyrazole of Formula (IV). This compound is halogenated (e.g., with bromine) in acetic acid or NBS in an inert solvent to give the bromopyrazole intermediate of Formula (V). A palladium catalyzed coupling reaction of (V) with a heteroarylboronic acid derivative of Formula (VI) provides the compounds of the invention of Formula (Ia) where X is other than CO₂H. A hydrolysis step of (Ia) provides the remaining compounds of the invention of Formula (Ib) where X is CO₂H.

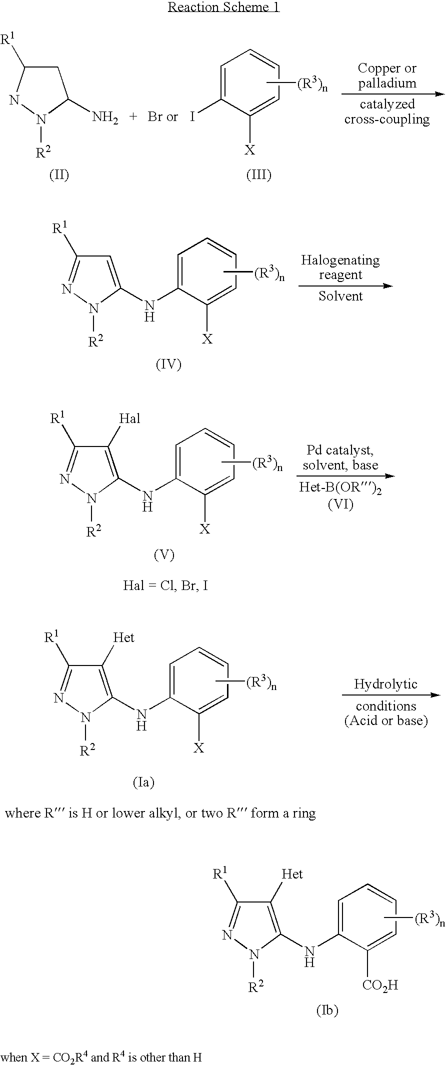

Elaboration of other compounds of the invention where one of the R³ substituents is a aryl or heteroaryl radical, Formula (Id), may be prepared from compounds of Formula (Ic) in which one of the R³ groups is halo, such as Cl or Br, using palladium coupling chemistry as illustrated in Reaction Scheme 2.

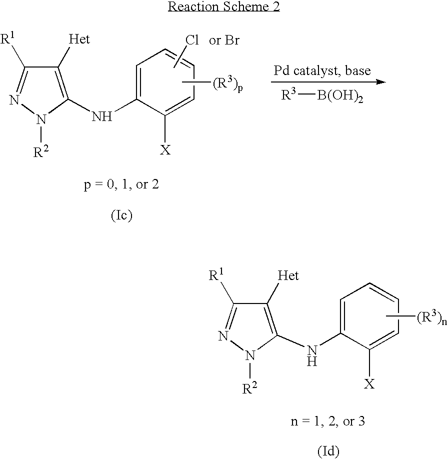

Other compounds of Formula (I) in which one of the R³ groups is NR⁴R⁴ or imidazol-1-yl may be prepared as shown in Reaction Scheme 3, from a compound of Formula (Ie) by a nucleophilic aromatic substitution reaction, facilitated by base.

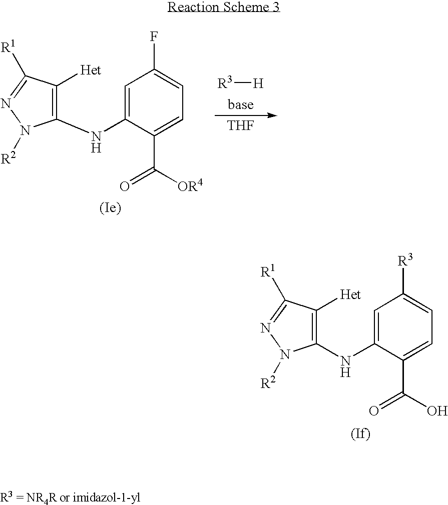

Compounds of the invention where Het is an oxazolyl radical, Formula (Ig, Ih), may be prepared from compounds of Formula (VIl). Conversion of the C-4 nitrile using hydrolytic condition provides to the corresponding amide Formula (VIl). Subsequent condensation with an appropriate electrophile (e.g., Formula (IX) and (X)), provides compounds of Formula (Ig) where X is other than CO₂H. A hydrolysis step of (Ig) provides the remaining compounds of the invention of Formula (Ih) where X is CO₂H.

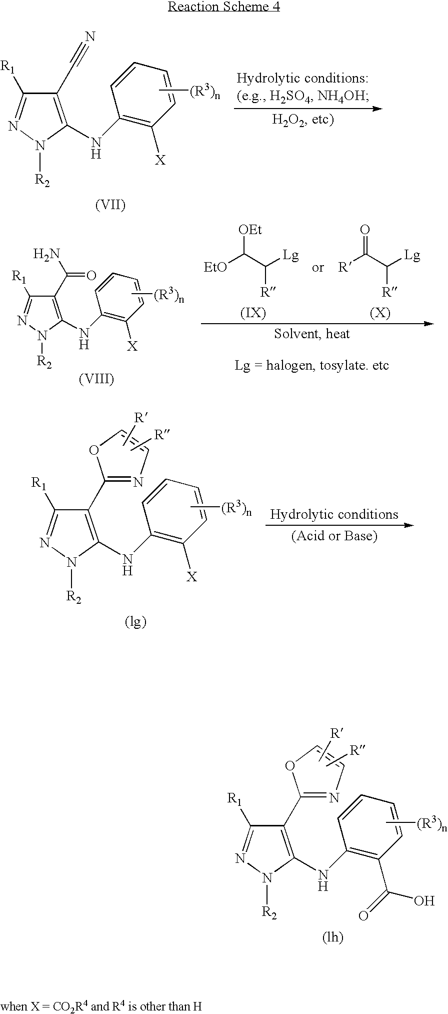

Compounds of the invention where Het is a thiazolyl radical, Formula (Ii, Ij), may be prepared from compounds of Formula (VII). Conversion of the nitrile group to the corresponding thioamide using a suitable reagent (e.g., H₂S, Lawessown's, dithiophosphates). Subsequent condensation with an appropriate electrophile (e.g., Formula (IX), (X), and (XII)) provide compounds of Formula (Ii) where X is other than CO₂H. A hydrolysis step of (ii) provides the remaining compounds of the invention of Formula (Ij) where X is CO₂H. 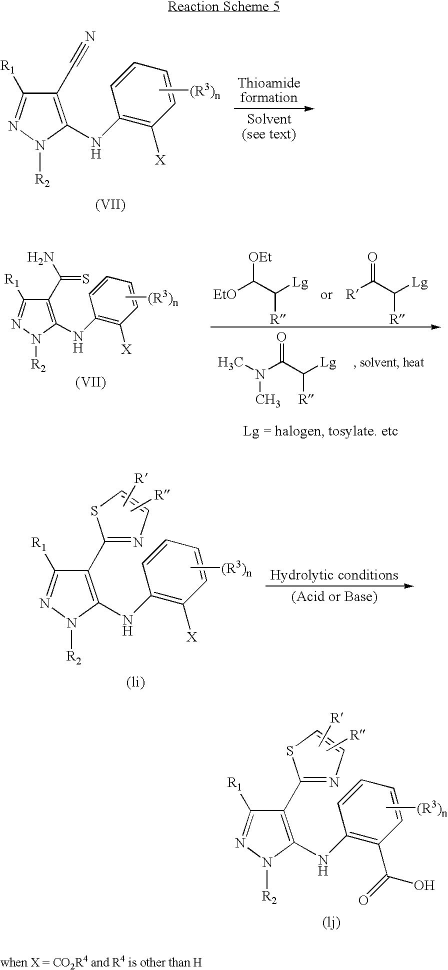
Synthesis of Intermediates

Intermediates are either commercially available, or are prepared by standard methods known in the art and/or by analogy to one of the procedures as described in U.S. patent application Ser. No. 10/719,485; filed Nov. 21, 2003.

The preparation of heterocyclic boronic acid derivatives used in Reaction Scheme 1 is illustrated in Reaction Scheme 6. A bromo or iodo-substituted heterocycle of Formula (XIII) can be treated with a organblithium and the resulting lithiated intermediate allowed to react with a boronate of Formula (R′″O)₃B to give the desired heterocyclic boronic acid derivative of Formula (VI). Alternatively, palladium catalyzed coupling of pinnacol borane (XIV) to (XIII) provides the corresponding Formula (VI) compound.

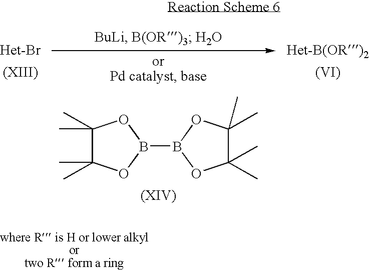

The preparation of 4-cyano pyrazoles (used in Reaction Schemes 4 and 5) can be achieved through the reaction sequence highlighted below (Reaction Scheme 7). The condensation of an orthoester (XV) with malontrile (XVI) provides compound (XVII), which can in turn be condensed with a substituted hydrazone to afford the aminopyrazole (XVIII). Subsequent palladium or copper mediated cross-coupling with an aryl halide (III) provides the desired intermediate (VII).

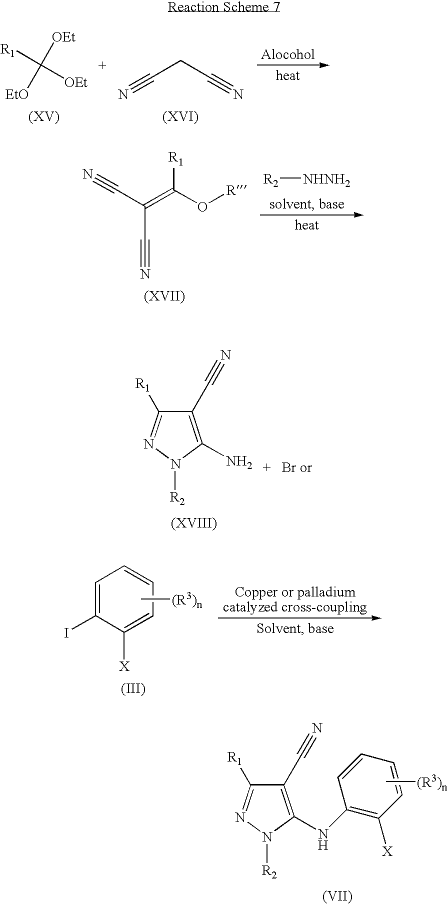

The present invention includes the prodrugs and salts of the prodrugs. Formation of prodrugs is well known in the art in order to enhance the properties of the parent compound; such properties include solubility, absorption, biostability, and release time (see, e.g., “Pharmaceutical Dosage Form and Drug Delivery Systems” (Sixth Edition), edited by Ansel et al., publ. by Williams & Wilkins, pgs. 27-29, (1995), which is hereby incorporated by reference). Commonly used prodrugs are designed to take advantage of the major drug biotransformation reactions, and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation, and acetylation (see, e.g., Goodman and Gilman's The Pharmacological Basis of Therapeutics (Ninth Edition), editor Molinoff et al., publ. by McGraw-Hill, pages 11-13, (1996), which is hereby incorporated by reference).

Salts of the compounds identified herein can be obtained by isolating the compounds as hydrochloride salts, prepared by treatment of the free base with anhydrous HCl in a suitable solvent such as THF. Generally, a desired salt of a compound of this invention can be prepared in situ during the final isolation and purification of a compound by means well known in the art; or a desired salt can be prepared by separately reacting the purified compound in its free base form with a suitable organic or inorganic acid and isolating the salt thus formed. These methods are conventional and would be readily apparent to one skilled in the art.

Additionally, sensitive or reactive groups on the compound of this invention may need to be protected and deprotected during any of the above methods. Protecting groups in general may be added and removed by conventional methods well known in the art (see, for example, T. W. Greene and P. G. M. Wuts, Protective Groups in Organic Synthesis; Wiley: New York, (1999).

A comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

For purposes of this invention, the chemical elements are identified in accordance with the Periodic Table of the Elements, CAS version, Handbook of Chemistry and Physics, 67th Ed., 1986-87.

Abbreviations and Acronyms

When the following abbreviations are used throughout the disclosure, they have the following meaning:
abs absolute
Ac acetyl
AcOH acetic acid
amu atomic mass unit
aq aqueous
BINAP 2,2′-Bis(diphenylphosphino)-1,1′-binaphthyl
Bn benzyl
Boc t-butoxycarbonyl
BTMAlCl₂ benzyltrimethylammonium dichloriodate
Bu butyl
CDCl₃ deuterochloroform
CDI carbonyl diimidazole
Celite® brand of diatomaceous earth filtering agent, registered trademark of Celite Corporation
CI-MS chemical ionization mass spectroscopy
conc concentrated
d doublet
DCM dichloromethane
dd doublet of doublet
ddd doublet of doublet of doublet
DMAP 4-(N,N-dimethyl)amino pyridine
DMF N,N-dimethyl formamide
DMSO dimethylsulfoxide
DMSO-d₆ dimethylsulfoxide-d₆
DOWEX® 66 Dowex hydroxide, weakly basic anion, macroporous, 25-50 mesh
dppf 1,1′-bis(diphenylphosphino)ferrocene
EDCl 1-(3-dimethylaminopropyl)-3-ethylcarbodiimide hydrochloride
EI electron impact ionization
EI-MS electron impact-mass spectrometry
equiv equivalent
ES-MS electrospray mass spectrometry
Et ethyl
Et₂O diethyl ether
Et₃N triethylamine
EtOAc ethyl acetate
EtOH ethanol
g gram
GC-MS gas chromatography-mass spectrometry
h hour(s)
Hex hexanes
¹H NMR proton nuclear magnetic resonance
HPLC high-performance liquid chromatography
HPLC ES-MS high-performance liquid chromatography-electrospray mass spectroscopy
KOtBu potassium tert-butoxide
L liter
LC-MS liquid chromatography/mass spectroscopy
LDA lithium diisopropylamide
Lg Leaving group (e.g., Cl, Br, I, tosylate, mesylate, triflate)
m multiplet
M molar
mL milliliter
m/z mass over charge
Me methyl
MeCN acetonitrile
MeOH methanol
mg milligram
MHz megahertz
min minute(s)
mmol millimole
mol mole
mp melting point
MS mass spectrometry
N normal
NaOAc sodium acetate
NBS N-bromosuccinimide
NIS N-iodosuccinimide
NMM 4-methylmorpholine
NMR nuclear magnetic resonance
Pd₂(dba)₃ tris(dibenzylideneacetone)dipalladium(0)
Pd(OAc)₂ palladium acetate
Pd(PPh₃)₄ tetrakis(triphenylphosphine)palladium(0)
Pd/C palladium on carbon
Pd(dppf)Cl₂ [1,1′-bis(diphenylphosphino)ferrocene]dichloropalladium(II)
Ph phenyl
ppm parts per million
Pr propyl
psi pounds per square inch
q quartet
qt quintet
rt room temperature
RT retention time (HPLC)
s singlet
satd saturated
TFA trifluoroacetic acid
THF tetrahydrofuran
TLC thin layer chromatography
TMS tetramethylsilane
v/v volume per unit volume
vol volume
w/w weight per unit weight
   General Experimental Methods

Air and moisture sensitive liquids and solutions were transferred via syringe or cannula, and introduced into reaction vessels through rubber septa. Commercial grade reagents and solvents were used without further purification. The term “concentration under reduced pressure” refers to use of a Buchi rotary evaporator at approximately 15 mm of Hg. All temperatures are reported uncorrected in degrees Celsius (° C.). Thin layer chromatography (TLC) was performed on EM Science pre-coated glass-backed silica gel 60 A F-254 250 μm plates. Column chromatography (flash chromatography) was performed on a Biotage system using 32-63 micron, 60 A, silica gel pre-packed cartridges. Purification using preparative reversed-phase HPLC chromatography were accomplished using a Gilson 215 system, typically using a YMC Pro-C18 AS-342 (150×20 mm I.D.) column. Typically, the mobile phase used was a mixture of H₂O (A) and MeCN (B). The water could be mixed or not with 0.1% TFA. A typical gradient was:

| Time | | | Flow |
|---|---|---|---|
| [min] | A: % | B: % | [mL/min] |
| 0.50 | 90.0 | 10.0 | 1.0 |
| 11.00 | 0.0 | 100.0 | 1.0 |
| 14.00 | 0.0 | 100.0 | 1.0 |
| 15.02 | 100.0 | 0.0 | 1.0 |

Electron impact mass spectra (EI-MS) were obtained with a Hewlett Packard 5989A mass spectrometer equipped with a Hewlett Packard 5890 Gas Chromatograph with a J & W DB-5 column (0.25 μM coating; 30 m×0.25 mm). The ion source was maintained at 250° C. and spectra were scanned from 50-800 amu at 2 sec per scan.

High pressure liquid chromatography-electrospray mass spectra (LC-MS) were obtained using either a:

(A) Hewlett-Packard 1100 HPLC equipped with a quaternary pump, a variable wavelength detector set at 254 nm, a YMC pro C-18 column (2×23 mm, 120 A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA, and B: 2% water in acetonitrile with 0.018% TFA. Gradient elution from 10% to 95% B over 3.5 minutes at a flow rate of 1.0 mL/min was used with an initial hold of 0.5 minutes and a final hold at 95% B of 0.5 minutes. Total run time was 6.5 minutes.

or

(B) Gilson HPLC system equipped with two Gilson 306 pumps, a Gilson 215 Autosampler, a Gilson diode array detector, a YMC Pro C-18 column (2×23 mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data was also acquired as an analog channel. The eluents were A: 2% acetonitrile in water with 0.02% TFA, and B: 2% water in acetonitrile with 0.018% TFA. Gradient elution from 10% to 90% B over 3.5 minutes at a flow rate of 1.5 mL/min was used with an initial hold of 0.5 minutes and a final hold at 90% B of 0.5 minutes. Total run time was 4.8 minutes. An extra switching valve was used for column switching and regeneration.

Routine one-dimensional NMR spectroscopy was performed on 300/400 MHz Varian Mercury-plus spectrometers. The samples were dissolved in deuterated solvents obtained from Cambridge Isotope Labs, and transferred to 5 mm ID Wilmad NMR tubes. The spectra were acquired at 293 K. The chemical shifts were recorded on the ppm scale and were referenced to the appropriate solvent signals, such as 2.49 ppm for DMSO-d₆, 1.93 ppm for CD₃CN, 3.30 ppm for CD₃OD, 5.32 ppm for CD₂Cl₂ and 7.26 ppm for CDCl₃ for ¹H spectra, and 39.5 ppm for DMSO-d₆, 1.3 ppm for CD₃CN, 49.0 ppm for CD₃OD, 53.8 ppm for CD₂Cl₂ and 77.0 ppm for CDCl₃ for ¹³C spectra.

### Preparation of Intermediates

### Intermediate A

### Preparation of Methyl 5-Bromo-2-{[3-tert-butyl-1-(2-methylphenyl)-1H-pyrazol-5-yl]amino}benzoate

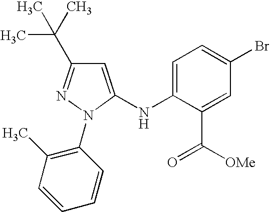

To a dried 25 mL flask was introduced 3-tert-butyl-1-(2-methylphenyl)-1H-pyrazol-5-amine (220 mg, 0.96 mmol), methyl 2,5-dibromobenzoate (235 mg, 0.80 mmol), Pd₂(dba)₃ (36.6 mg, 0.04 mmol), BINAP (49.8 mg, 0.08 mmol), and Cs₂CO₃ (365 mg, 1.12 mmol). The flask was degassed followed by addition of toluene (1 mL), and the mixture was then heated to 110° C. for 20 h. The mixture was cooled to rt, and diluted with ethyl acetate. The solid was filtered off, and the solvent was removed under reduced pressure. The residue was redissolved in methanol/THF (4:1, v/v) and filtered though a C₈-silica plug. HPLC purification using a gradient elution from 10% to 90% acetonitrile in water afforded 110 mg (31%) of the title compound. ¹H NMR (300 MHz, CD₂Cl₂) δ 9.21 (s, 1 H), 7.41 (d, 1 H), 7.20-7.30 (m, 5 H), 7.10 (d, 1 H), 6.09 (s, 1 H), 3.72 (s, 3 H), 2.04 (s, 3 H), 1.30 (s, 9 H). ES-MS m/z 444.1 (MH⁺); HPLC RT (min) 4.30.

### Intermediate B

### Preparation of Methyl 2-[(4-Bromo-3-tert-butyl-1-methyl-1H-pyrazol-5-yl)amino]-5-methoxybenzoate

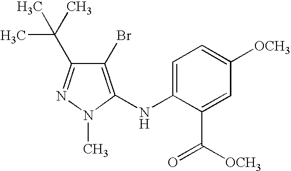

To a solution of 2-(5-tert-butyl-2-methyl-2H-pyrazol-3-ylamino)-5-methoxy-benzoic acid methyl ester (1.34 g, 4.22 mmol) in acetic acid (27 mL), was added dropwise a solution of Br₂ (6.74 g, 4.22 mmol) in acetic acid (5 mL). The reaction was stirred for 5 min, and then water (100 mL) was added. The aqueous phase was extracted with EtOAc, and the combined organic layers were washed with water, and then with NaHCO₃ (10% aqueous solution, 10 times). The organic layer was then dried (Na₂SO₄), filtered, and concentrated under reduced pressure. The residue was purified by silica gel flash chromatography (eluent: 5 to 10% EtOAc in hexane) to give the title compound as a light yellow solid (1.49 g, 89%). ¹H NMR (400 MHz, CDCl₃) δ 1.40 (s, 9H), 3.66 (s, 3H), 3.78 (s, 3H), 4.05 (s, 3H), 6.32 (d, 1H), 7.06 (dd, 1H), 7.48 (d, 1H).

### Intermediate C

### Preparation of Methyl 2-{[4-Bromo-3-methyl-1-(2-methylphenyl)-1H-pyrazol-5-yl]amino}-5-methoxybenzoate

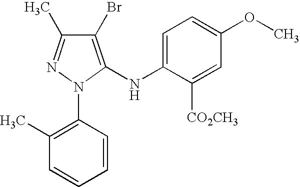

To a solution of methyl 5-methoxy-2-{[3-methyl-1-(2-methylphenyl)-1H-pyrazol-5-yl]amino}benzoate (1.4 g, 3.87 mmol) in AcOH (20 mL) was added 618 mg (3.87 mmol) Br₂. The reaction was stirred at rt for 3 h, and then water was added. The precipitate was collected by filtration, and then redissolved in EtOAc. The solution was washed sequentially with NaHCO₃ (10% aqueous solution) and water. The organic phase was dried (Na₂SO₄), filtered, and concentrated under reduced pressure. The residue was purified by flash silica gel column chromatography (eluent: 5 to 10% EtOAc in hexane) to give the title compound (1.2 g, 72%) as a yellow solid. ¹H NMR (400 MHz, acetone-d₆) δ 2.16 (s, 3H), 2.29 (s, 3H), 3.74 (s, 3H), 3.84 (s, 3H), 6.62 (d, 1H), 7.06 (dd, 1H), 7.21-7.25 (m, 1H), 7.28-7.32 (m, 3H), 7.35 (d, 1H).

### Intermediate D

### Preparation of 2-{[4-Iodo-1-(2-methylphenyl)-3-(4-methylphenyl)-1H-pyrazol-5-yl]amino}-5-methylbenzoic Acid

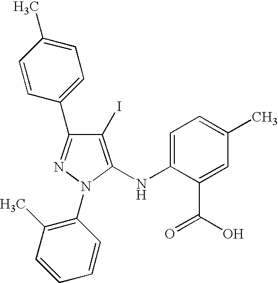

To a solution of 5-methyl-2-{[1-(2-methylphenyl)-3-(4-methylphenyl)-1H-pyrazol-5-yl]amino}benzoic acid (49.5 mg, 0.13 mmol) in AcOH/DCM (1:1, v/v) (2 mL) was added a solution of NIS (28 mg, 0.13 mmol) in DCM (1 mL). The reaction was stirred at rt for 3 h. Water (1 mL) was added to the reaction mixture. The water layer was extracted with DCM (2 mL), and the combined organic layers were washed sequentially with 10% aqueous sodium sulfite solution and brine, and then concentrated under reduced pressure. The crude product was subjected to preparative HPLC purification with a gradient elution from 30% to 95% acetonitrile in water to afford 9.1 mg (14%) of the title compound. ¹H NMR (300 MHz, CD₂Cl₂) δ 8.88 (s, 1 H), 7.85 (d, 2 H), 7.74 (s, 1 H), 7.18-7.32 (m, 7 H), 6.58 (d, 1 H), 2.41 (s, 3 H), 2 25 (s, 3 H), 2 21 (s, 3 H). ES-MS m/z 524.1 (MH⁺); HPLC RT (min) 4.35.

### SPECIFIC EXAMPLES OF THE INVENTION

By using the above general methods and procedures analogous thereto, compounds of the invention may be made. The following specific examples are presented to further illustrate the invention described herein, but should not be construed as limiting the scope of the invention in any way.

### Preparation of the Invention Compounds

### Example 1

### Preparation of 5-Methoxy-2-{[3-methyl-1-(2-methylphenyl)-4-(3-thienyl)-1H-pyrazol-5-yl]amino}benzoic Acid

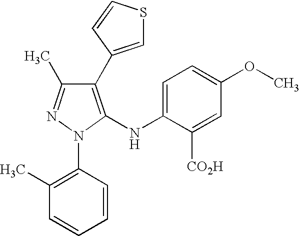

Through a mixture of methyl 2-{[4-bromo-3-methyl-1-(2-methylphenyl)-1H-pyrazol-5-yl]amino}-5-methoxybenzoate (Intermediate C, 100 mg, 0.23 mmol), 3-thiopheneboronic acid (118 mg, 0.93 mmol), Pd(PPh₃)₄ (16 mg, 1.16 mmol) and Na₂CO₃ (0.58 mL, 2 M solution in water) in 2.5 mL DMF was passed a flow of N₂ for 15 min. The mixture was sealed in a Emrys™ Process Vials (size M) with a crimp top and heated in a microwave reactor (Emrys™ Optimizer) at 150° C. for 15 min. The reaction mixture was cooled to rt and filtered. The filtrate was concentrated, and the residue dissolved in a mixture of THF (2 mL), MeOH (1 mL) and water (2 mL). LiOH (55 mg) was added, and the mixture was stirred at 50° C. for 2 h and then at rt for 16 h. The reaction mixture was concentrated under reduced pressure and the residue purified by preparative HPLC. The desired fractions were concentrated under reduced pressure, and the residue was treated with NH₄Cl (saturated solution in water) and extracted with CH₂Cl₂. The combined organic layers were dried (Na₂SO₄), filtered, and concentrated under reduced pressure. The residue was further purified by silica gel column chromatography (eluent: 50 to 100% ethyl acetate in hexane) to give the title product (23.3 mg, 23%). ¹H NMR (400 MHz, acetone-d₆) δ 2.19 (s, 3H), 2.48 (s, 3H), 3.70 (s, 3H), 6.49 (d, 1H), 6.89 (dd, 1H), 7.19-7.21 (m, 1H), 7.7.26-7.30 (m, 4H), 7.35 (d, 1H), 7.39-7.41 (m, 2H). ES-MS m/z 420.2 (MH⁺); HPLC RT (min) 3.40.

### Example 2

### Preparation of 2-{[4-(3-Furyl)-3-methyl-1-(2-methylphenyl)-1H-pyrazol-5-yl]amino}-5-methoxybenzoic Acid

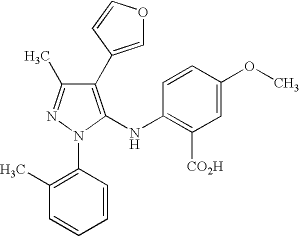

Through a mixture of methyl 2-{[4-bromo-3-methyl-1-(2-methylphenyl)-1H-pyrazol-5-yl]amino}-5-methoxybenzoate (Intermediate C, 100 mg, 0.23 mmol), 3-furanboronic acid (104 mg, 0.93 mmol), Pd(PPh₃)₄ (16 mg, 1.16 mmol), and Na₂CO₃ (0.58 mL, 2 M solution in water) in 2.5 mL DMF was passed a flow of N₂ for 15 min. The mixture was sealed in a Emrys™ Process Vials (size M) with a crimp top and heated in a microwave reactor (Emrys™ Optimizer) at 150° C. for 15 min. The reaction mixture was cooled to rt and filtered. The filtrate was concentrated, and the residue dissolved in a mixture of THF (2 mL), MeOH (1 mL) and water (2 mL). LiOH (55 mg) was added, and the mixture was stirred at 50° C. for 2 h and then at rt for 16 h. The reaction mixture was then concentrated under reduced pressure, and the residue purified by preparative HPLC. The desired fractions were concentrated under reduced pressure, and the residue was treated with NH₄Cl (saturated solution in water) and extracted with CH₂Cl₂. The combined organic layers were dried (Na₂SO₄), filtered, and concentrated under reduced pressure. The residue was further purified by silica gel column chromatography (eluent: 50 to 100% ethyl acetate in hexane) to give the title product (6.4 mg, 6.8%). ¹H NMR (400 MHz, acetone-d₆) δ 2.18 (s, 3H), 2.44 (s, 3H), 3.71 (s, 3H), 6.49 (d, 1H), 6.6 (t, 1H), 6.69 (dd, 1H), 7.18-7.21 (m, 1H), 7.26-7.28 (m, 3H), 7.38 (d, 1H), 7.50 (t, 1H), 7.66 (s, 1H). ES-MS m/z 404.1 (MH⁺); HPLC RT (min) 3.28.

### Example 3

### Preparation of 2-{[4-(1-Benzothien-2-yl)-3-methyl-1-(2-methylphenyl)-1H-pyrazol-5-yl]amino}-5-methoxybenzoic Acid

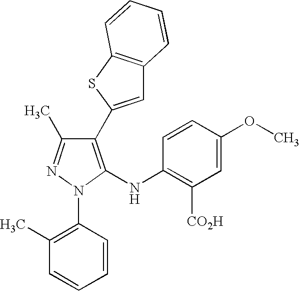

Through a mixture of methyl 2-{[4-bromo-3-methyl-1-(2-methylphenyl)-1H-pyrazol-5-yl]amino}-5-methoxybenzoate (Intermediate C, 100 mg, 0.23 mmol), benzo(B) thiophene-2-boronic acid (165 mg, 0.93 mmol), Pd(PPh₃)₄ (16 mg, 1.16 mmol), and Na₂CO₃ (0.58 mL, 2 M solution in water) in 2.5 mL DMF was passed a flow of N₂ for 15 min. The mixture was sealed in a Emrys™ Process Vials (size M) with a crimp top and heated in a microwave reactor (Emrys™ Optimizer) at 150° C. for 15 min. The reaction mixture was cooled to rt and filtered. The filtrate was concentrated, and the residue dissolved in a mixture of THF (2 mL), MeOH (1 mL) and water (2 mL). LiOH (55 mg) was added, and the mixture was stirred at 50° C. for 2 h and then at rt for 16 h. The desired fractions were concentrated under reduced pressure, and the residue was purified by HPLC. The residue was treated with NH₄Cl (satd solution in water) and extracted with CH₂Cl₂. The combined organic layers were dried (Na₂SO₄), filtered, and concentrated under reduced pressure to give the title product (39.7 mg, 36%). ¹H NMR (400 MHz, acetone-d₆) δ 2.21 (s, 3H), 2.60 (s, 3H), 3.68 (s, 3H), 6.54 (d, 1H), 6.89 (dd, 1H), 7.24-7.33 (m, 6H), 7.37 (d, 1H), 7.49 (s, 1H), 7.75-7.80 (m, 2H). ES-MS m/z 470.2 (MH⁺); HPLC RT (min) 3.77.

### Example 4

### Preparation 5-methoxy-2-{[1-(2-methoxyphenyl)-1′,3-dimethyl-1H,1′H-4,4′-bipyrazol-5-yl]amino}benzoic acid

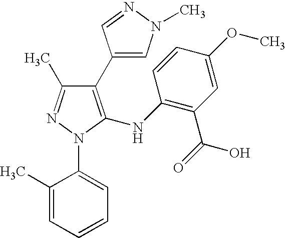

To a mixture of methyl 2-{[4-bromo-3-methyl-1-(2-methylphenyl)-1H-pyrazol-5-yl]amino}-5-methoxybenzoate (Intermediate C, 0.5 g, 1.16 mmol) and 1-methyl-4(4,4,5,5,-tetramethyl-1,3,2-oxaborolan-2-yl)-1H-pyrazole (0.36 g, 1.74 mmol) in toluene (9 mL) was added ethanol (3 mL) followed by NaHCO₃ saturated solution (3 mL). The resulting suspension was degassed using a flow of nitrogen gas for 15 min, and then PdCl₂(dppf)₂ (0.09 g, 0.11 mmol) was added and the mixture was heated at 80° C. for 6 h. The reaction mixture was diluted with ethyl acetate and filtered through Celite® and concentrated. The residue was purified by silica gel column chromatography eluting with hexane/EtOAc 10%-20% gradient. The resulting solid was dissolved in MeOH (6 mL) and 1N NaOH was added. The mixture was heated at 55° C. overnight, cooled to rt, and concentrated under reduced pressure. The residue was taken up in water and acidified, the precipitate was filtered and washed with water. The resulting white solid was suspended in MeOH (2 mL), sonicated for 10 min, and filtered to give the desired product (0.07 g, 21%). ¹H NMR (400 MHz, CD₃OD) δ 2.15 (s, 3H), 2.43 (s, 3H), 3.66 (s, 3H), 3.81 (s, 3H), 6.44 (d, 2H), 6.83 (dd, 2H), 7.11-7.23 (m, 1H), 7.24-7.33 (m, 1H), 7.37 (d, 1H), 7.46 (s, 1H), 7.65 (s, 1H). ES-MS m/z 418.2 (MH⁺); RT (min) 2.82.

Using the methods analogous to those described above in Reaction Schemes 1-3 and 6, and in Examples 1-4, and by selecting the appropriate starting materials (e.g., compounds of Formula (VI) in Reaction Scheme 6), additional compounds of the invention may also be prepared. These compounds are illustrated in Table 1 below.

TABLE 1

| Example | | |
|---|---|---|
| No. | Structure | IUPAC Name |
| 5 | 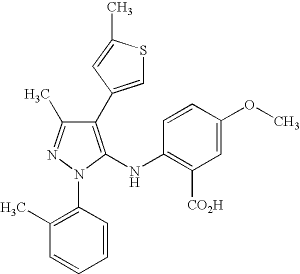 | 5-methoxy-2-{[3-methyl-1-(2-methylphenyl)- 4-(5-methyl-3-thienyl)-1H-pyrazol-5- yl]amino}benzoic acid |
| 6 | 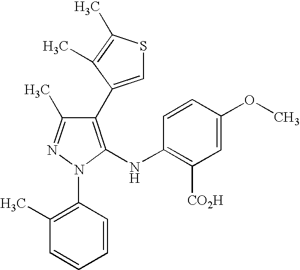 | 2-{[4-(4,5-dimethyl-3-thienyl)-3-methyl-1-(2- methylphenyl)-1H-pyrazol-5-yl]amino}-5- methoxybenzoic acid |
| 7 | 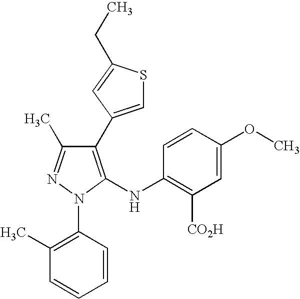 | 2-{[4-(5-ethyl-3-thienyl)-3-methyl-1-(2- methylphenyl)-1H-pyrazol-5-yl]amino}-5- methoxybenzoic acid |
| 8 | 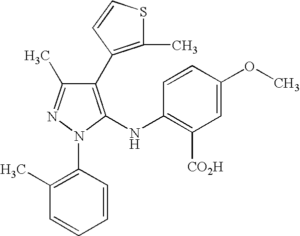 | 5-methoxy-2-{[3-methyl-1-(2-methylpheyl)- 4-(2-methyl-3-thienyl)-1H-pyrazol-5- yl]amino}benzoic acid |
| 9 | 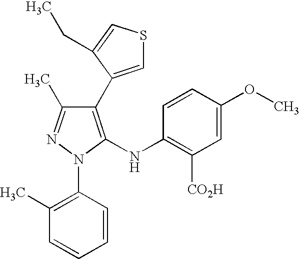 | 2-{[4-(4-ethyl-3-thienyl)-3-methyl-1-(2- methylphenyl)-1H-pyrazol-5-yl]amino}-5- methoxybenzoic acid |
| 10 | 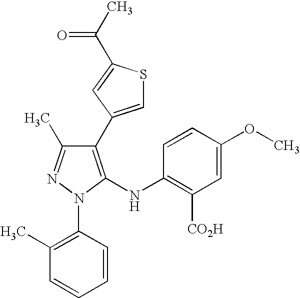 | 2-{[4-(5-acetyl-3-thienyl)-3-methyl-1-(2- methylphenyl)-1H-pyrazol-5-yl]amino}-5- methoxybenzoic acid |
| 11 | 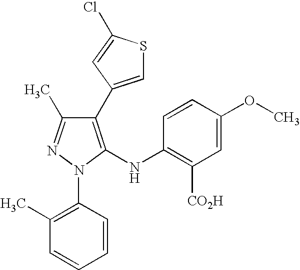 | 2-{[4-(5-chloro-3-thienyl)-3-methyl-1-(2- methylphenyl)-1H-pyrazol-5-yl]amino}-5- methoxybenzoic acid |
| 12 | 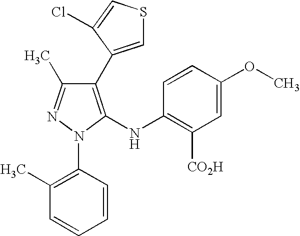 | 2-{[4-(4-chloro-3-thienyl)-3-methyl-1-(2- methylphenyl)-1H-pyrazol-5-yl]amino}-5- methoxybenzoic acid |
| 13 | 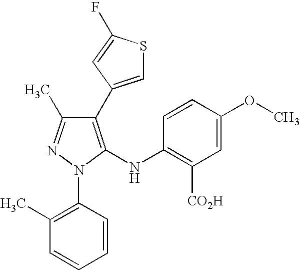 | 2-{[4-(5-fluoro-3-thienyl)-3-methyl-1-(2- methylphenyl)-1H-pyrazol-5-yl]amino}-5- methoxybenzoic acid |
| 14 | 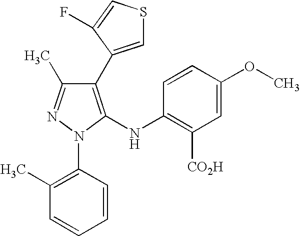 | 2-{[4-(4-fluoro-3-thienyl)-3-methyl-1-(2- methylphenyl)-1H-pyrazol-5-yl]amino}-5- methoxybenzoic acid |
| 15 | 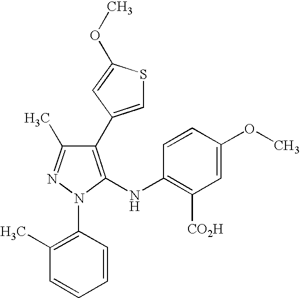 | 5-methoxy-2-{[4-(5-methoxy-3-thienyl)-3- methyl-1-(2-methylphenyl)-1H-pyrazol-5- yl]amino}benzoic acid |
| 16 | 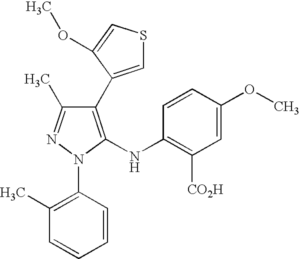 | 5-methoxy-2-{[4-(5-methoxy-3-thienyl)-3- methyl-1-(2-yl]amino}benzoic acid |
| 17 | 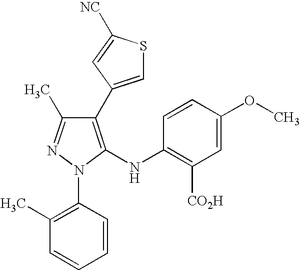 | 2-{[4-(5-cyano-3-thienyl)-3-methyl-1-(2- methylphenyl)-1H-pyrazol-5-yl]amino}-5- methoxybenzoic acid |
| 18 | 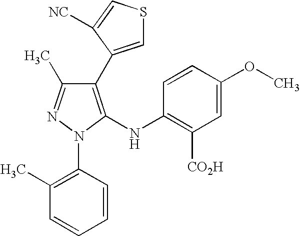 | 2-{[4-(4-cyano-3-thienyl)-3-methyl-1-(2- methylphenyl)-1H-pyrazol-5-yl]amino}-5- methoxybenzoic acid |
| 19 | 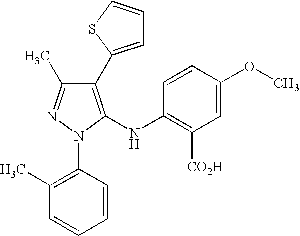 | 5-methoxy-2-{[3-methyl-1-(2-methylphenyl)- 4-(2-thienyl)-1H-pyrazol-5-yl]amino}benzoic acid |
| 20 | 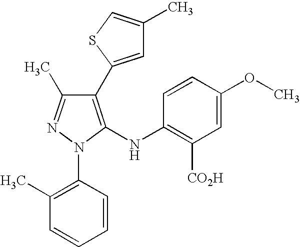 | 5-methoxy-2-{[3-methyl-1-(2-methylphenyl)- 4-(4-methyl-2-thienyl)-1H-pyrazol-5- yl]amino}benzoic acid |
| 21 | 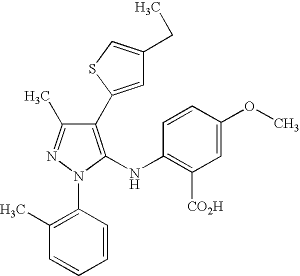 | 2-{[4-(4-ethyl-2-thienyl)-3-methyl-1-(2- methylphenyl)-1H-pyrazol-5-yl]amino}-5- methoxybenzoic acid |
| 22 | 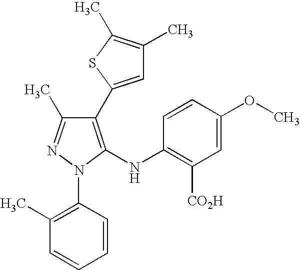 | 2-{[4-(4,5-dimethyl-2-thienyl)-3-methyl-1-(2- methylphenyl)-1H-pyrazol-5-yl]amino}-5- methoxybenzoic acid |
| 23 | 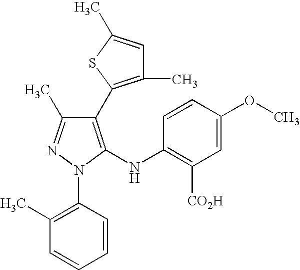 | 2-{[4-(3,5-dimethyl-2-thienyl)-3-methyl-1-(2- methylphenyl)-1H-pyrazol-5-yl]amino}-5- methoxybenzoic acid |
| 24 | 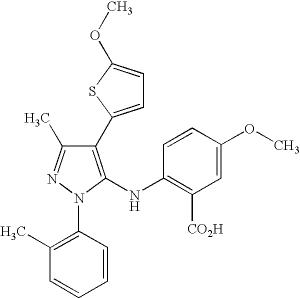 | 5-methoxy-2-{[4-(5-methoxy-2-thienyl)-3- methyl-1-(2-methylphenyl)-1H-pyrazol-5- yl]amino}benzoic acid |
| 25 | 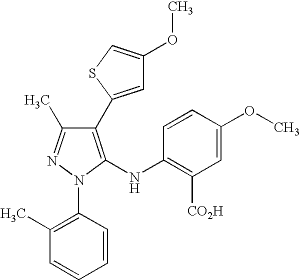 | 5-methoxy-2-{[4-(4-methoxy-2-thienyl)-3- methyl-1-(2-methylphenyl)-1H-pyrazol-5- yl]amino}benzoic acid |
| 26 | 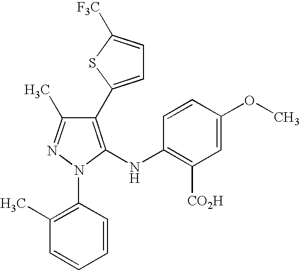 | 5-methoxy-2-({3-methyl-1-(2-methylphenyl)- 4-[5-(trifluoromethyl)-2-thienyl]-1H-pyrazol-5- yl}amino)benzoic acid |
| 27 | 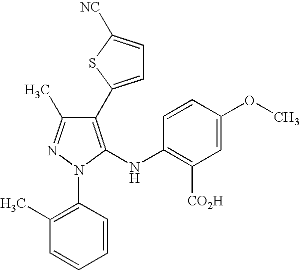 | 2-{[4-(5-cyano-2-thienyl)-3-methyl-1-(2- methylphenyl)-1H-pyrazol-5-yl]amino}-5- methoxybenzoic acid |
| 28 | 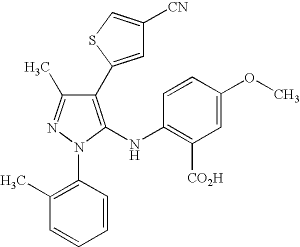 | 2-{[4-(4-cyano-2-thienyl)-3-methyl-1-(2- methylphenyl)-1H-pyrazol-5-yl]amino}-5- methoxybenzoic acid |
| 29 | 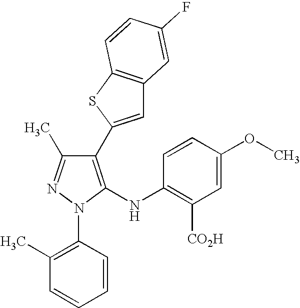 | 2-{[4-(5-fluoro-1-benzothien-2-yl)-3-methyl-1- (2-methylphenyl)-1H-pyrazol-5-yl]amino}-5- methoxybenzoic acid |
| 30 | 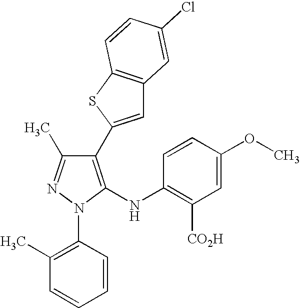 | 2-{[4-(5-chloro-1-benzothien-2-yl)-3-methyl- 1-(2-methylphenyl)-1H-pyrazol-5-yl]amino}-5- methoxybenzoic acid |
| 31 | 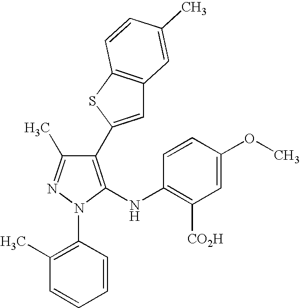 | 5-methoxy-2-{[3-methyl-4-(5-methyl-1- benzothien-2-yl)-1-(2-methylphenyl)-1H- pyrazol-5-yl]amino}benzoic acid |
| 32 | 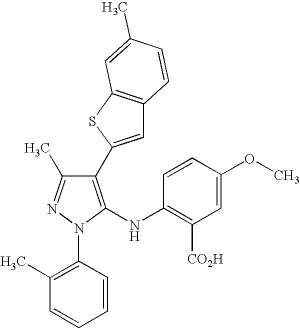 | 5-methoxy-2-{[3-methyl-4-(6-methyl-1- benzothien-2-yl)-1-(2-methylphenyl)-1H- pyrazol-5-yl]amino}benzoic acid |
| 33 | 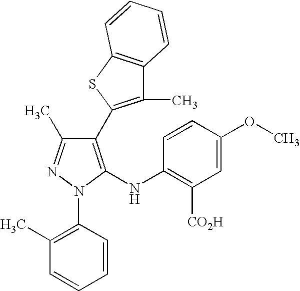 | 5-methoxy-2-{[3-methyl-4-(3-methyl-1- benzothien-2-yl)-1-(2-methylphenyl)-1H- pyrazol-5-yl]amino}benzoic acid |
| 34 | 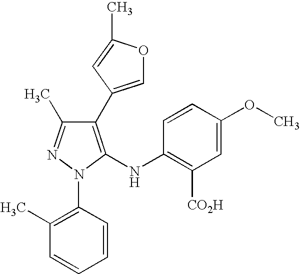 | 5-methoxy-2-{[3-methyl-4-(5-methyl-3-furyl)- 1-(2-methylphenyl)-1H-pyrazol-5- yl]amino}benzoic acid |
| 35 | 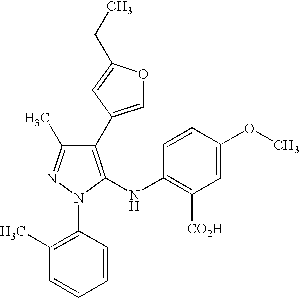 | 2-{[4-(5-ethyl-3-furyl)-3-methyl-1-(2- methylphenyl)-1H-pyrazol-5-yl]amino}-5- methoxybenzoic acid |
| 36 | 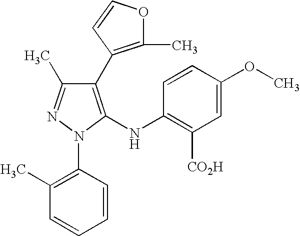 | 5-methoxy-2-{[3-methyl-4-(2-methyl-3-furyl)- 1-(2-methylphenyl)-1H-pyrazol-5- yl]amino}benzoic acid |
| 37 | 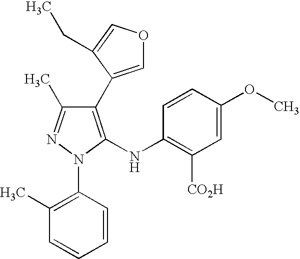 | 2-{[4-(4-ethyl-3-furyl)-3-methyl-1-(2- methylphenyl)-1H-pyrazol-5-yl]amino}-5- methoxybenzoic acid |
| 38 | 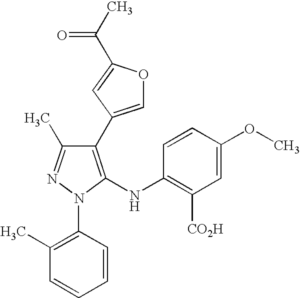 | 2-{[4-(5-acetyl-3-furyl)-3-methyl-1-(2- methylphenyl)-1H-pyrazol-5-yl]amino}-5- methoxybenzoic acid |
| 39 | 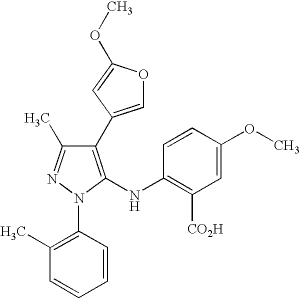 | 5-methoxy-2-{[4-(5-methoxy-3-furyl)-3- methyl-1-(2-methylphenyl)-1H-pyrazol-5- yl]amino}benzoic acid |
| 40 | 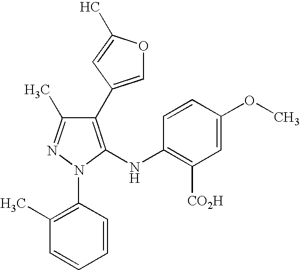 | 2-{[4-(5-cyano-3-furyl)-3-methyl-1-(2- methylphenyl)-1H-pyrazol-5-yl]amino}-5- methoxybenzoic acid |
| 41 | 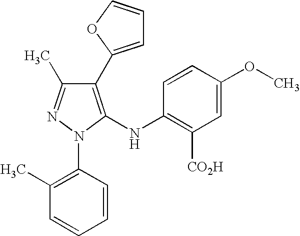 | 2-{[4-(2-furyl)-3-methyl-1-(2-methylphenyl)- 1H-pyrazol-5-yl]amino}-5-methoxybenzoic acid |
| 42 | 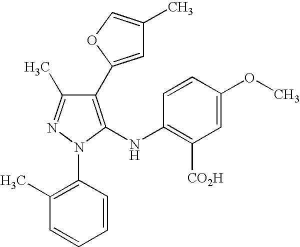 | 5-methoxy-2-{[3-methyl-4-(4-methyl-2-furyl)- 1-(2-methylphenyl)-1H-pyrazol-5- yl]amino}benzoic acid |
| 43 | 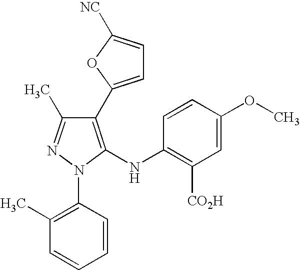 | 2-{[4-(5-cyano-2-furyl)-3-methyl-1-(2- methylphenyl)-1H-pyrazol |
| 44 | 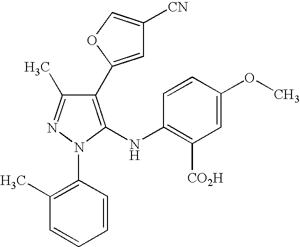 | 2-{[4-(4-cyano-2-furyl)-3-methyl-1-(2- methylphenyl)-1H-pyrazol-5-yl]amino}-5- methoxybenzoic acid |
| 45 | 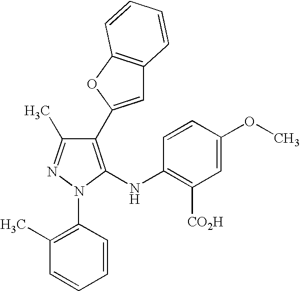 | 2-{[4-(1-benzofuran-2-yl)-3-methyl-1-(2- methylphenyl)-1H-pyrazol-5-yl]amino}-5- methoxybenzoic acid |
| 46 | 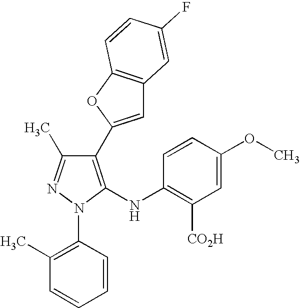 | 2-{[4-(5-fluoro-1-benzofuran-2-yl)-3-methyl-1- (2-methylphenyl)-1H-pyrazol-5-yl]amino}-5- methoxybenzoic acid |
| 47 | 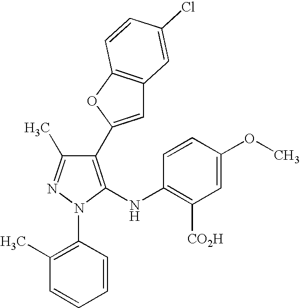 | 2-{[4-(5-chloro-1-benzofuran-2-yl)-3-methyl- 1-(2-methylphenyl)-1H-pyrazol-5-yl]amino}-5- methoxybenzoic acid |
| 48 | 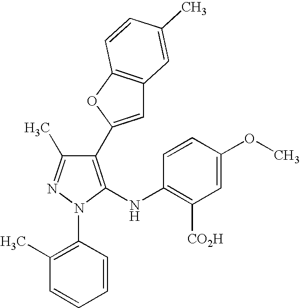 | 5-methoxy-2-{[3-methyl-4-(5-methyl-1- benzofuran-2-yl)-1-(2-methylphenyl)-1H- pyrazol-5-yl]amino}benzoic acid |
| 49 | 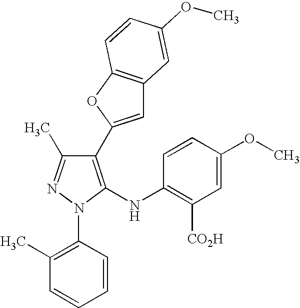 | 5-methoxy-2-{[4-(5-methoxy-1-benzofuran-2- yl)-3-methyl-1-(2-methylphenyl)-1H-pyrazol 5-yl]amino}benzoic acid |
| 50 | 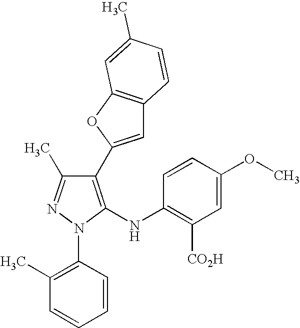 | 5-methoxy-2-{[3-methyl-4-(6-methyl-1- benzofuran-2-yl)-1-(2-methylphenyl)-1H- pyrazol-5-yl]amino}benzoic acid |
| 51 | 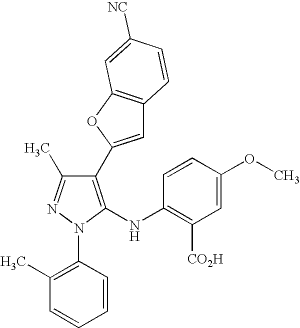 | 2-{[4-(6-cyano-1-benzofuran-2-yl)-3-methyl- 1-(2-methylphenyl)-1H-pyrazol-5-yl]amino}-5- methoxybenzoic acid |
| 52 | 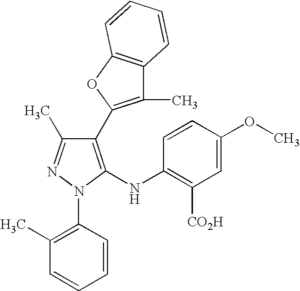 | 5-methoxy-2-{[3-methyl-4-(3-methyl-1- benzofuran-2-yl)-1-(2-methylphenyl)-1H- pyrazol-5-yl]amino}benzoic acid |
| 53 | 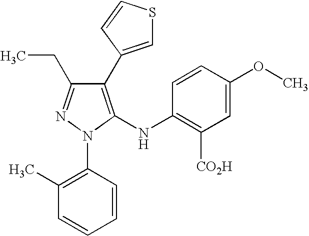 | 2-{[3-ethyl-1-(2-methylphenyl)-4-(3-thienyl)- 1H-pyrazol-5-yl]amino}-5-methoxybenzoic acid |
| 54 | 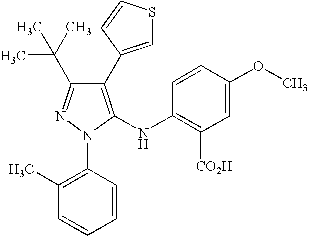 | 2-{[3-tert-butyl-1-(2-methylphenyl)-4-(3- thienyl)-1H-pyrazol-5-yl]amino}-5- methoxybenzoic acid |
| 55 | 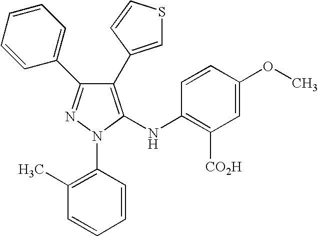 | 5-methoxy-2-{[1-(2-methylphenyl)-3-phenyl- 4-(3-thienyl)-1H-pyrazol-5-yl]amino}benzoic acid |
| 56 | 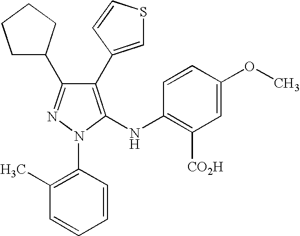 | 2-{[3-cyclopentyl-1-(2-methylphenyl)-4-(3- thienyl)-1H-pyrazol-5-yl]amino}-5- methoxybenzoic acid |
| 57 | 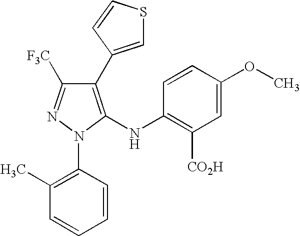 | 5-methoxy-2-{[1-(2-methylphenyl)-4-(3- thienyl)-3-(trifluoromethyl)-1H-pyrazol-5- yl]amino}benzoic acid |
| 58 | 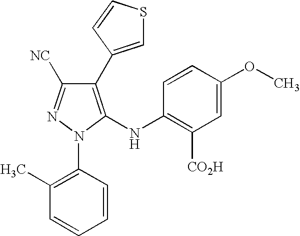 | 2-{[3-cyano-1-(2-methylphenyl)-4-(3-thienyl)- 1H-pyrazol-5-yl]amino}-5-methoxybenzoic acid |
| 59 | 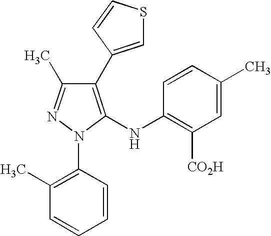 | 5-methyl-2-{[3-methyl-1-(2-methylphenyl)-4- (3-thienyl)-1H-pyrazol-5-yl]amino}benzoic acid |
| 60 | 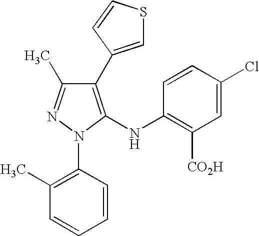 | 5-chloro-2-{[3-methyl-1-(2-methylphenyl)-4- (3-thienyl)-1H-pyrazol-5-yl]amino}benzoic acid |
| 61 | 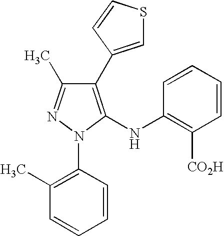 | 2-{[3-methyl-1-(2-methylphenyl)-4-(3-thienyl)- 1H-pyrazol-5-yl]amino}benzoic acid |
| 62 | 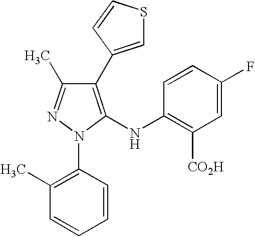 | 5-fluoro-2-{[3-methyl-1-(2-methylphenyl)-4- (3-thienyl)-1H-pyrazol-5-yl]amino}benzoic acid |
| 63 | 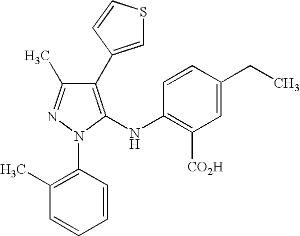 | 5-ethyl-2-{[3-methyl-1-(2-methylphenyl)-4-(3- thienyl)-1H-pyrazol-5-yl]amino}benzoic acid |
| 64 | 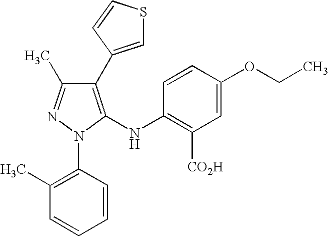 | 5-ethoxy-2-{[3-methyl-1-(2-methylphenyl)-4- (3-thienyl)-1H-pyrazol-5-yl]amino}benzoic acid |
| 65 | 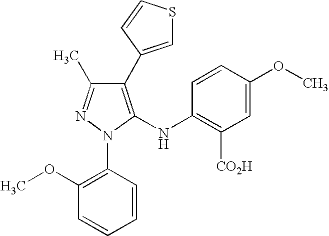 | 5-methoxy-2-{[1-(2-methoxyphenyl)-3- methyl-4-(3-thienyl)-1H-pyrazol-5- yl]amino}benzoic acid |
| 66 | 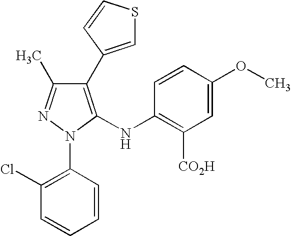 | 2-{[1-(2-chlorophenyl)-3-methyl-4-(3-thienyl)- 1H-pyrazol-5-yl]amino}-5-methoxybenzoic acid |
| 67 | 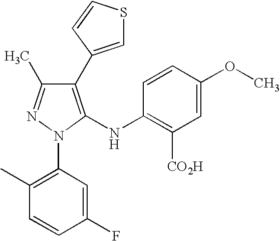 | 2-{[1-(5-fluoro-2-methylphenyl)-3-methyl-4- (3-thienyl)-1H-pyrazol-5-yl]amino}-5- methoxybenzoic acid |
| 68 | 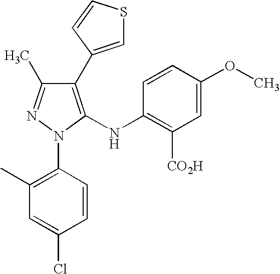 | 2-{[1-(4-chloro-2-methylphenyl)-3-methyl-4- (3-thienyl)-1H-pyrazol-5-yl]amino}-5- methoxybenzoic acid |
| 69 | 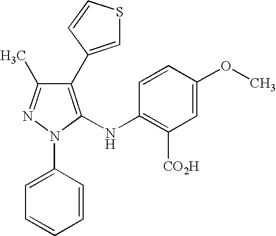 | 5-methoxy-2-{[3-methyl-1-phenyl-4-(3- thienyl)-1H-pyrazol-5-yl]amino}benzoic acid |
| 70 | 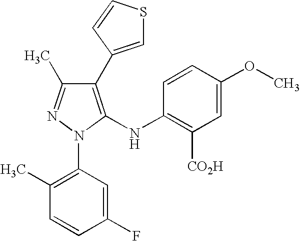 | 2-{[1-(5-fluoro-2-methylphenyl)-3-methyl-4- (3-thienyl)-1H-pyrazol-5-yl]amino}-5- methoxybenzoic acid |
| 71 | 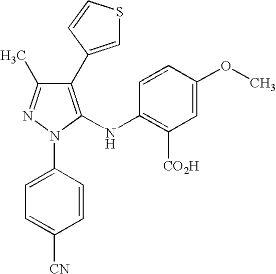 | 2-{[1-(4-cyanophenyl)-3-methyl-4-(3-thienyl)- 1H-pyrazol-5-yl]amino}-5-methoxybenzoic acid |
| 72 | 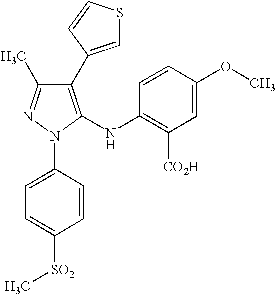 | 5-methoxy-2-{[3-methyl-1-[4- (methylsulfonyl)phenyl]-4-(3-thienyl)-1H- pyrazol-5-yl]amino}benzoic acid |
| 73 | 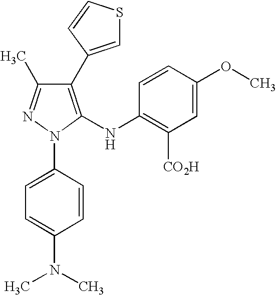 | 2-{[1-[4-(dimethylamino)phenyl]-3-methyl-4- (3-thienyl)-1H-pyrazol-5-yl]amino}-5- methoxybenzoic acid |
| 74 | 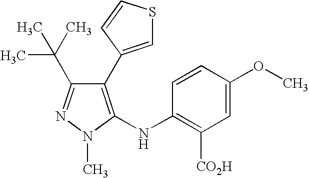 | 2-{[3-tert-butyl-1-methyl-4-(3-thienyl)-1H- pyrazol-5-yl]amino}-5-methoxybenzoic acid |
| 75 | 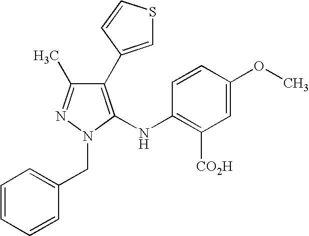 | 2-{[1-benzyl-3-methyl-4-(3-thienyl)-1H- pyrazol-5-yl]amino}- |
| 76 | 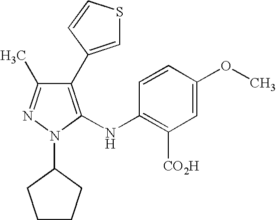 | 2-{[1-cyclopentyl-3-methyl-4-(3-thienyl)-1H- pyrazol-5-yl]amino}-5-methoxybenzoic acid |
| 77 | 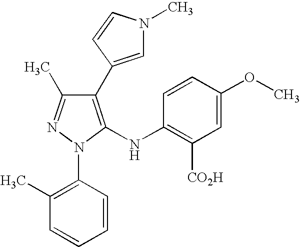 | 5-methoxy-2-{[3-methyl-1-(2-methylphenyl)- 4-(1-methyl-1H-pyrrol-3-yl)-1H-pyrazol-5- yl]amino}benzoic acid |
| 78 | 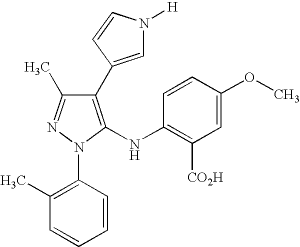 | 5-methoxy-2-{[3-methyl-1-(2-methylphenyl)- 4-(1H-pyrrol-3-yl)-1H-pyrazol-5- yl]amino}benzoic acid |
| 79 | 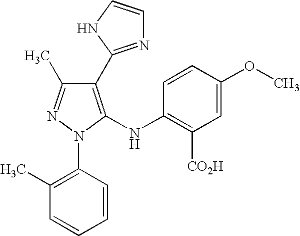 | 2-{[4-(1H-imidazol-2-yl)-3-methyl-1-(2- methylphenyl)-1H-pyrazol-5-yl]amino}-5- methoxybenzoic acid |
| 80 | 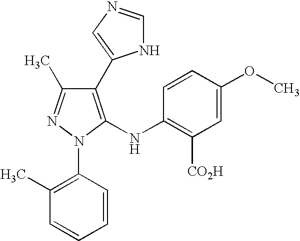 | 2-{[4-(1H-imidazol-5-yl)-3-methyl-1-(2- methylphenyl)-1H-pyrazol-5-yl]amino}-5- methoxybenzoic acid |
| 81 | 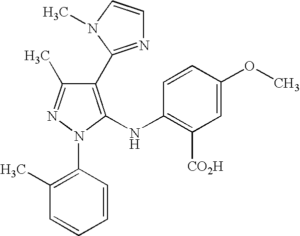 | 5-methoxy-2-{[3-methyl-4-(1-methyl-1H- imidazol-2-yl)-1-(2-methylphenyl)-1H- pyrazol-5-yl]amino}benzoic acid |
| 82 | 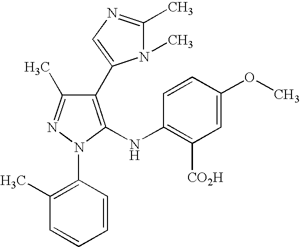 | 2-{[4-(1,2-dimethyl-1H-imidazol-5-yl)-3- methyl-1-(2-methylphenyl)-1H-pyrazol-5- yl]amino}-5-methoxybenzoic acid |
| 83 | 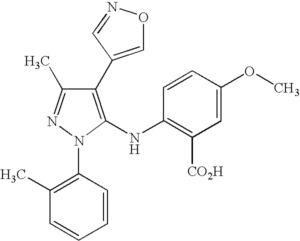 | 2-{[4-isoxazol-4-yl-3-methyl-1-(2- methylphenyl)-1H-pyrazol-5-yl]amino}-5- methoxybenzoic acid |
| 84 | 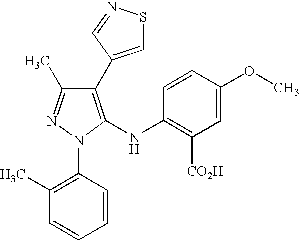 | 2-{[4-isothiazol-4-yl-3-methyl-1-(2- methylphenyl)-1H-pyrazol-5-yl]amino}-5- methoxybenzoic acid |
| 85 | 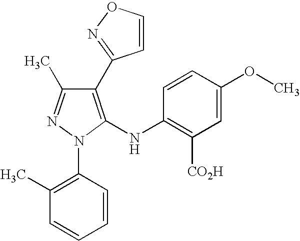 | 2-{[4-isoxazol-3-yl-3-methyl-1-(2- methylphenyl)-1H-pyrazol-5-yl]amino}-5- methoxybenzoic acid |
| 86 | 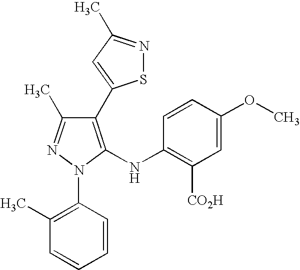 | 5-methoxy-2-{[3-methyl-4-(3- methylisothiazol-5-yl)-1-(2-methylphenyl)-1H- pyrazol-5-yl]amino}benzoic acid |
| 87 | 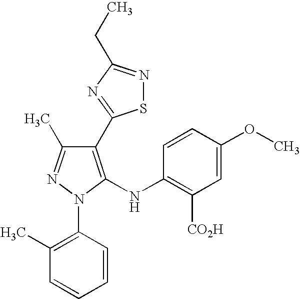 | 2-{[4-(3-ethyl-1,2,4-thiadiazol-5-yl)-3-methyl- 1-(2-methylphenyl)-1H-pyrazol-5-yl]amino}-5- methoxybenzoic acid |
| 88 | 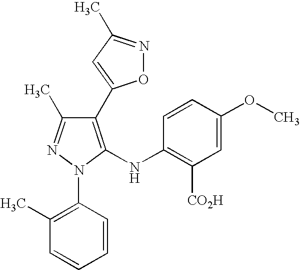 | 5-methoxy-2-{[3-methyl-4-(3-methylisoxazol- 5-yl)-1-(2-methylphenyl)-1H-pyrazol-5- yl]amino}benzoic acid |

### Example 89

### Preparation of 5-Amino-3-methyl-1-o-tolyl-1H-pyrazole-4-carbonitrile

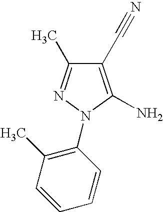

1-Ethoxymethylenemalonitrile (3.76 g, 27.09 mmol) was carefully added to a solution of 2-methylphenylhydrazine hydrochloride (4.43 g, 27.09 mmol) and triethylamine (3.93 mL, 27.09 mmol) in ethanol (25 mL). The mixture was then refluxed overnight and cooled to room temperature. The resulting suspension was taken up in dichloromethane and washed with water, dried over anhydrous sodium sulfate, and concentrated. The residue was taken up in hexanes and the suspension was filtered and the orange solid dried (4.72 g, 82%). ES-MS m/z 213.2 (MH⁺); HPLC RT (min) 2.16.

### Example 90

### Preparation of 2-(4-Cyano-5-methyl-2-o-tolyl-2H-pyrazol-3-ylamino)-5-methoxy-benzoic acid methyl ester

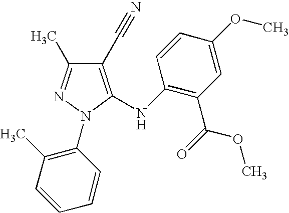

To a mixture of 5-amino-3-methyl-1-o-tolyl-1H-pyrazole-4-carbonitrile (3.0 g, 14.13 mmol), 2-bromo-5-methoxy-benzoic acid methyl ester (Example 89, 2.89 g, 11.78 mmol) in toluene (35 mL) was added BINAP (0.73 g, 1.18 mmol) followed by tris(dibenzylideneacetone) dipalladium (0.65 g, 0.71 mmol). To the mixture was added cesium carbonate (5.37 g, 16.49 mmol), and the suspension was heated at 118° C. overnight. The reaction mixture was then cooled to rt, diluted with ethyl acetate, filtered through Celite® and the filtrate concentrated under reduced pressure. The residue was purified by silica gel column chromatography (25% ethyl acetate-hexanes) to give a pale yellow solid (3.32 g, 75%). ES-MS m/z 377.1 (MH⁺); HPLC RT (min) 3.35.

### Example 91

### Preparation of 2-(4-carbamoyl-5-methyl-2-o-tolyl-2H-pyrazol-3-ylamino)-5-methoxy-benzoic acid methyl ester

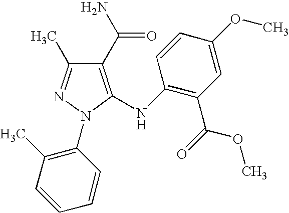

2-(4-Cyano-5-methyl-2-o-tolyl-2H-pyrazol-3-ylamino)-5-methoxy-benzoic acid methyl ester (Example 90, 1.20 g, 3.19 mmol) was added in portion to sulfuric acid (10 mL) on at rt. The suspension was stirred at rt until completion (2.5 days, monitored by LC-MS). The reaction mixture was poured onto crushed ice and neutralized with concentrated ammonium hydroxide. The resultant suspension was filtered to give a light brown solid (1.02 g, 81%). ES-MS m/z 395.3 (MH⁺); HPLC RT (min) 2.72.

### Example 92

### Preparation of 5-Methoxy-2-(5-methyl-4-oxazol-2-yl-2-o-tolyl-2H-pyrazol-3-ylamino)-benzoic acid methyl ester

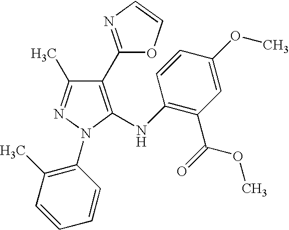

A mixture of 2-(4-carbamoyl-5-methyl-2-o-tolyl-2H-pyrazol-3-ylamino)-5-methoxy-benzoic acid methyl ester (Example 91, 0.1 g, 0.25 mmol) and 2-bromo-1,1-diethoxy-ethane (0.07 g, 0.38 mmol) in dioxane (1.0 mL)/toluene (1.0 mL) was heated at 125° C. for 6 h and concentrated. The product (0.054 g, 51%) was isolated by HPLC using Waters C-18 column (30 to 80% acetonitrile/water). ¹H NMR (400 MHz, CD₂Cl₂) δ 2.16 (s, 3H), 2.57 (s, 3H), 3.67 (s, 3H), 3.84 (s, 3H), 6.53 (d, 1H), 6.75-6.79 (m, 1H), 7.15 (s, 1H), 7.28-7.31 (m, 5H), 7.75 (s, 1H). ES-MS m/z 419.1 (MH⁺); HPLC RT (min) 3.30.

### Example 93

### Preparation of 5-Methoxy-2-(5-methyl-4-oxazol-2-yl-2-o-tolyl-2H-pyrazol-3-ylamino)-benzoic acid

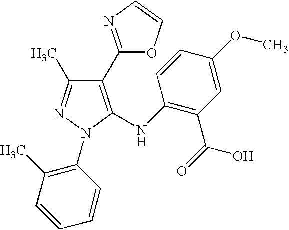

A mixture of 5-methoxy-2-(5-methyl-4-oxazol-2-yl-2-o-tolyl-2H-pyrazol-3-ylamino)-benzoic acid methyl ester (Example 92, 0.40 g, 0.10 mmol) and LiOH (0.2 g, 0.96 mmol) in THF (2.0 mL), methanol (1.0 mL), and water (1.0 mL) was stirred at rt for 18 h and concentrated. The residue was taken up in water and acidified, and the resulting precipitate was collected by filtration. The product (0.026 g, 66%) was isolated by HPLC using waters C-18 column (15 to 80% acetonitrile/water). ¹H NMR (400 MHz, CD₂Cl₂) δ 2.16 (s, 3H), 2.63 (s, 3H), 3.72 (s, 3H), 6.64 (d, 1H), 6.72-6.79 (m, 1H), 7.21-7.33 (m, 5H), 7.41 (s, 1H), 8.00 (s, 1H). ES-MS m/z 405.1 (MH⁺); HPLC RT (min) 2.91.

Reaction Scheme 8 summarizes the experimentals described in Examples 89-93.

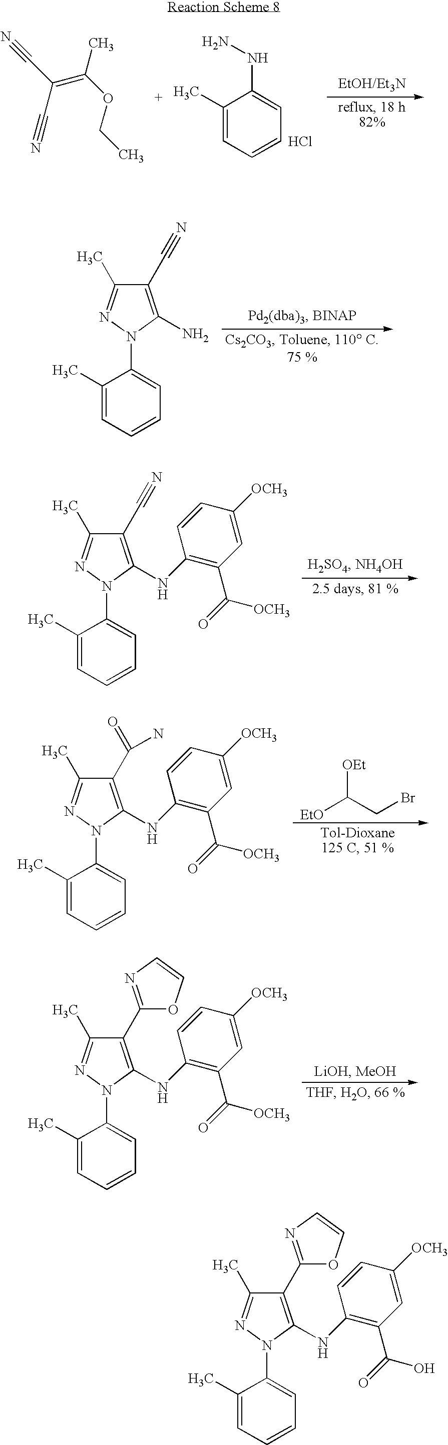

Using the methods analogous to those described above in Reaction Schemes 4, 7, and 8 and in Examples 89-93, and by applying the appropriate starting materials, additional compounds of the invention may also be prepared. Examples of such compounds are illustrated in Table 2 below.

TABLE 2

| Example | | LC-MS | |
|---|---|---|---|
| No. | Structure | Data | IUPAC Name |
| 94 | 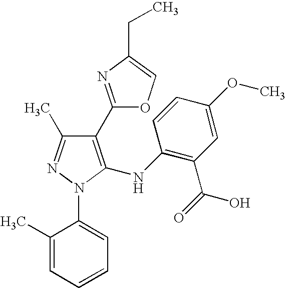 | RT = 3.23 MIN, MH+ = 433.1 | 2-[4-(4-Ethyl-oxazol-2-yl)-5- methyl-2-o-tolyl-2H-pyrazol-3- ylamino]-5-methoxy-benzoic acid |
| 95 | 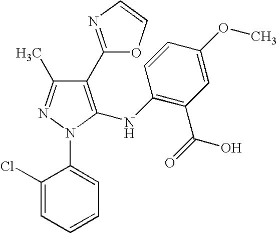 | RT = 2.90 MIN, M+ = 425.1 | 2-[2-(2-Chloro-phenyl)-5- methyl-4-oxazol-2-yl-2H- pyrazol-3-ylamino]-5-methoxy- benzoic acid |
| 96 | 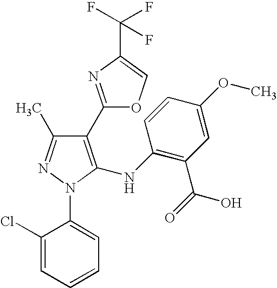 | RT = 3.42 MIN, M+ = 493.1 | 2-[2-(2-Chloro-phenyl)-5- methyl-4-(4-trifluoromethyl- oxazol-2-yl)-2H-pyrazol-3- ylamino]-5-methoxy-benzoicacid |
| 97 | 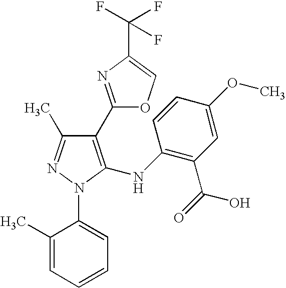 | RT = 3.45 MIN, MH+ = 473.1 | 5-Methoxy-2-[5-methyl-2-o- tolyl-4-(4-trifluoromethyl- oxazol-2-yl)-2H-pyrazol-3- ylamino]-benzoic acid |

### Example 98

### Preparation of 2-(1-Ethoxy-propylidene)-malononitrile

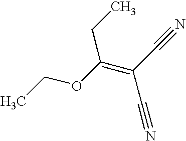

A mixture of triethyl orthopropionate (7.0 g, 38.52 mmol) and malononitrile (2.57 g, 38.52 mmol) was heated to reflux for 5 h and cooled to rt. The reaction mixture was used in the next step without further workup or purification.

### Example 99

### Preparation of 5-Amino-3-ethyl-1-(2-methoxy-phenyl)-1H-pyrazole-4-carbonitrile

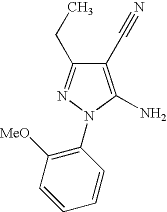

2-(1-Ethoxy-propylidene)-malononitrile (Example 98, 2.98 g, 15.68 mmol) was carefully added to a solution of 2-methoxyphenylhydrazine (2.82 g, 15.68 mmol) and triethylamine (2.28 mL, 15.68 mmol) in ethanol (40 mL). The mixture was then refluxed for 18 h and cooled to rt. The resulting suspension was taken up in dichloromethane and washed with water, dried over anhydrous sodium sulfate, and concentrated. The residue was suspended in hexanes and filtered and the orange solid (3.37 g, 89%) dried on the high vacuum pump. ES-MS m/z 243.2 (MH⁺); HPLC RT (min) 2.32.

### Example 100

### Preparation of 3-Ethyl-1-(2-methoxy-phenyl)-5-p-tolylamino-1H-pyrazole-4-carbonitrile

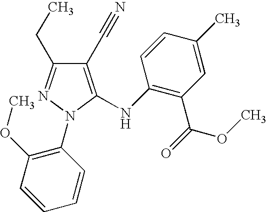

To a mixture of 5-amino-3-ethyl-1-(2-methoxy-phenyl)-1H-pyrazole-4-carbonitrile (Example 99, 1.0 g, 4.11 mmol), 2-bromo-5-methyl-benzoic acid methyl ester (0.78 g, 3.43 mmol) in toluene (25 mL) was added BINAP (0.21 g, 0.34 mmol) followed by tris(dibenzylideneacetone) dipalladium (0) (0.19 g, 0.21 mmol). To the mixture was added cesium carbonate (1.56 g, 4.80 mmol) and the suspension was heated at 118° C. overnight and cooled to rt. Then reaction mixture was diluted with ethyl acetate and filtered through Celite® and concentrated. The residue purified by silica gel column chromatography (25% ethyl acetate-hexanes) to give a pale yellow foamy solid (1.22 g, 91%). ES-MS m/z 391.1 (MH⁺); HPLC RT (min) 3.73.

### Example 101

### Preparation of 2-[5-Ethyl-2-(2-methoxy-phenyl)-4-thiocarbamoyl-2H-pyrazol-3-ylamino]-5-methyl-benzoic acid methyl ester

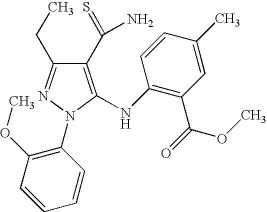

To a suspension of 3-ethyl-1-(2-methoxy-phenyl)-5-p-tolylamino-1H-pyrazole-4-carbonitrile (Example 100, 1.57 g, 4.02 mmol) in water (4 mL) was added O,O-diethyl dithiophosphate (3.0 mL, 16.1 mmol). The suspension was heated at 80° C. under nitrogen for 18 h and cooled to rt. The mixture was diluted with ethyl acetate and washed with water, dried over Na₂SO₄ and concentrated. The product was isolated by silica gel column chromatography (25% ethyl acetate-hexanes, then 100% ethyl acetate) to give a yellow solid (0.603 g, 35%). ES-MS m/z 425.1 (MH⁺); HPLC RT (min) 3.36.

### Example 102

### Preparation of 2-[5-ethyl-2-(2-methoxy-phenyl)-4-(4-methoxy-thiazol-2-yl)-2H-pyrazol-3-ylamino]-5-methyl-benzoic acid methyl ester

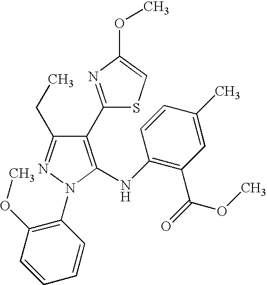

A mixture of 2-[5-ethyl-2-(2-methoxy-phenyl)-4-thiocarbamoyl-2H-pyrazol-3-ylamino]-5-methyl-benzoic acid methyl ester (Example 101, 0.603 g, 1.42 mmol) and 2-chloro-N,N-dimethylacetamide (0.229 g, 1.85 mmol) in methanol (20 mL) was heated at 70° C. for 4.5 h and concentrated in vacuo. The residue was purified by silica gel column chromatography (25% ethyl acetate in hexanes) to give a pale yellow solid (0.445 g, 65%). ¹H NMR (400 MHz, CD₂Cl₂) δ 1.34 (t, 3H), 2.14 (s, 3H), 3.08 (q, 2H), 3.85 (s, 6H), 3.87 (s, 3H), 6.10 (s, 1H), 6.33 (d, 1H), 6.95-7.11 (m, 3H), 7.32-7.40 (m, 2H), 7.62 (s, 1H), 9.22 (s, 1H). ES-MS m/z 479.1 (MH⁺); HPLC RT (min) 3.90.

### Example 103

### Preparation of 2-[5-ethyl-2-(2-methoxy-phenyl)-4-(4-methoxy-thiazol-2-yl)-2H-pyrazol-3-ylamino]-5-methyl-benzoic acid

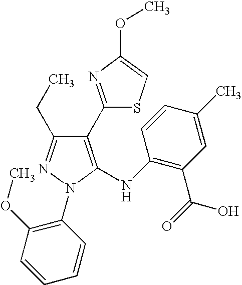

A mixture of 2-[5-ethyl-2-(2-methoxy-phenyl)-4-(4-methoxy-thiazol-2-yl)-2H-pyrazol-3-ylamino]-5-methyl-benzoic acid methyl ester (Example 102, 0.45 g, 0.94 mmol) and LiOH (0.23 g, 9.40 mmol) in THF (20 mL), methanol (10 mL), and water (10 mL) was stirred at 35° C. for 4 h and concentrated. The residue was taken up in water and acidified, then filtered. The solid was dried on the high vacuum pump to give a pale yellow solid (0.397 g, 91%). ¹H NMR (400 MHz, CD₃OD) δ 1.36 (t, 3H), 2.16 (s, 3H), 3.08 (q, 2H), 3.85 (s, 3H), 3.90 (s, 3H), 6.12 (s, 1H), 6.30 (d, 1H), 6.92-6.99 (m, 2H), 7.09 (d, 1H), 7.31-7.42 (m, 2H), 7.63 (s, 1H). ES-MS m/z 465.1 (MH⁺); HPLC RT (min) 3.45.

Reaction Scheme 9 summarizes the experimentals of Examples 98-103.

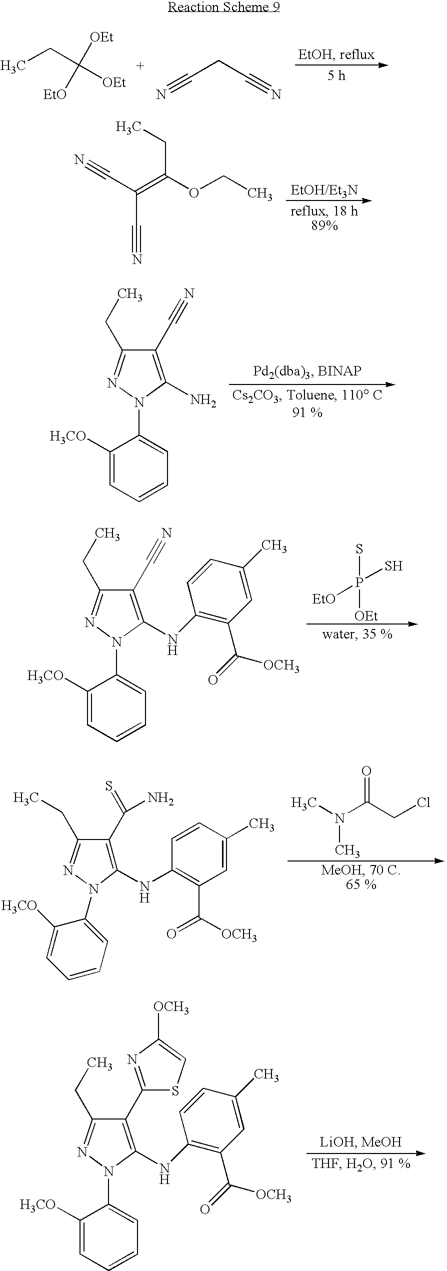

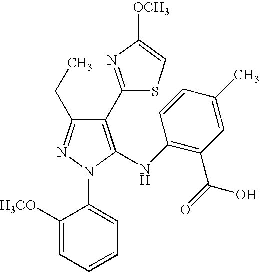

### Example 104

### Preparation of 2-[5-Ethyl-2-(2-methoxy-phenyl)-4-thiazol-2-yl-2H-pyrazol-3-ylamino]-5-methyl-benzoic acid methyl ester

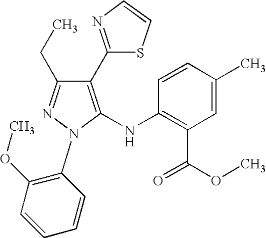

A mixture of 2-[5-ethyl-2-(2-methoxy-phenyl)-4-thiocarbamoyl-2H-pyrazol-3-ylamino]-5-methyl-benzoic acid methyl ester (Example 101, 0.03 g, 0.08 mmol) and 2-bromo-1,1-diethoxy-ethane (0.02 g, 0.10 mmol) in methanol (1.5 mL) was heated at 70° C. for 4.5 h and concentrated in vacuo. The product (0.012 g, 33%) was isolated by HPLC using waters C-18 column (55 to 90% acetonitrile/water). ¹H NMR (400 MHz, CD₂Cl₂) δ 1.35 (t, 3H), 2.16 (s, 3H), 3.08 (q, 2H), 3.85 (s, 3H), 3.87 (s, 3H), 6.32 (d, 1H), 6.95-7.11 (m, 2H), 7.17 (d, 1H), 7.37-7.42 (m, 3H), 7.62 (s, 1H), 9.22 (s, 1H). ES-MS m/z 449.1 (MH⁺); HPLC RT (min) 3.35.

### Example 105

### Preparation of 2-[5-Ethyl-2-(2-methoxy-phenyl)-4-thiazol-2-yl-2H-pyrazol-3-ylamino]-5-methyl-benzoic acid

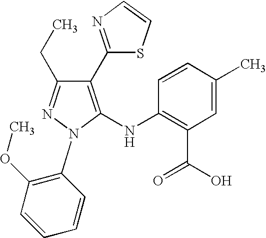

A mixture of 2-[5-ethyl-2-(2-methoxy-phenyl)-4-thiazol-2-yl-2H-pyrazol-3-ylamino]-5-methyl-benzoic acid methyl ester (Example 104, 0.012 g, 0.027 mmol) and LiOH (0.006 g, 0.27 mmol) in THF (2 mL), methanol (1 mL), and water (1 mL) was stirred at 35° C. for 1 h and concentrated. The product (0.009 g, 77%) was isolated by HPLC using waters C-18 column (15 to 80% acetonitrile/water). ¹H NMR (400 MHz, CD₃OD) δ 1.34 (t, 3H), 2.17 (s, 3H), 3.08 (q, 2H), 3.81 (s, 3H), 6.22 (d, 1H), 6.80 (d, 1H), 6.94-7.11 (m, 2H), 7.31-7.42 (m, 3H), 7.61 (s, 1H), 7.78 (d, 1H). ES-MS m/z 435.1 (MH⁺); HPLC RT (min) 3.30.

Reaction Scheme 10 summarizes the experimentals of Examples 104 and 105.

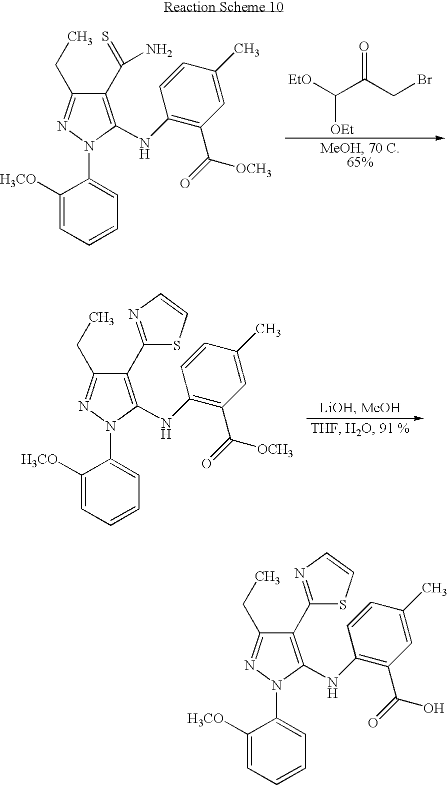

### Example 106

### Preparation of 2-[5-Ethyl-2-(2-methoxy-phenyl)-4-thiocarbamoyl-2H-pyrazol-3-ylamino]-5-methoxy-benzoic acid methyl ester

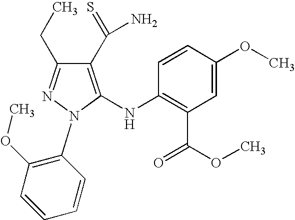

To a suspension of 3-ethyl-1-(2-methoxy-phenyl)-5-p-tolylamino-1H-pyrazole-4-carbonitrile (0.5 g, 1.23 mmol) which was prepared using methods similar to those used for the preparation of Example 90 and Example 100, in water (2 mL) was added O,O-diethyl dithiophosphate (0.55 mL, 16.08 mmol). The suspension was heated at 80° C. under nitrogen for 1 h and cooled to rt. The mixture was diluted with ethyl acetate and washed with water, dried over Na₂SO₄ and concentrated under reduced pressure. The product was isolated by silica gel column chromatography (25% ethyl acetate-hexanes, then 100% ethyl acetate) to give a yellow solid (0.24 g, 44%). ES-MS m/z 441.2 (MH⁺); HPLC RT (min) 3.13.

### Example 107

### Preparation of 2-[5-Ethyl-4-(4-ethyl-thiazol-2-yl)-2-(2-methoxy-phenyl)-2H-pyrazol-3-ylamino]-5-methoxy-benzoic acid methyl ester

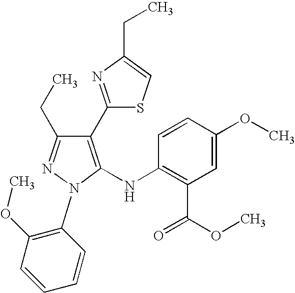

A mixture of 2-[5-ethyl-2-(2-methoxy-phenyl)-4-thiocarbamoyl-2H-pyrazol-3-ylamino]-5-methoxy-benzoic acid methyl ester (Example 106, 0.042 g, 0.095 mmol) and 1-bromo-butan-2-one (0.019 g, 0.124 mmol) in methanol (1.5 mL) was heated at 80° C. for 4.5 h and concentrated in vacuo. The product (0.039 g, 88%) was isolated by HPLC using waters C-18 column (55 to 90% acetonitrile/water). ¹H NMR (400 MHz, CD₃OD) δ 1.17 (t, 3H), 1.34 (t, 3H), 2.73 (q, 2H), 3.08 (q, 2H), 3.62 (s, 3H), 3.85 (s, 3H), 3.87 (s, 3H), 6.40 (d, 1H), 6.70 (d, 1H), 6.95 (s, 1H), 7.01-7.18 (m, 2H), 7.34-7.41 (m, 3H). ES-MS m/z 493.2 (MH⁺); HPLC RT (min) 3.48.

### Example 108

### Preparation of 2-[5-Ethyl-4-(4-ethyl-thiazol-2-yl)-2-(2-methoxy-phenyl)-2H-pyrazol-3-ylamino]-5-methoxy-benzoic acid

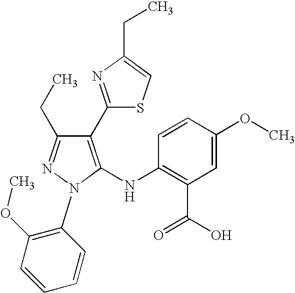

A mixture of 2-[5-ethyl-4-(4-ethyl-thiazol-2-yl)-2-(2-methoxy-phenyl)-2H-pyrazol-3-ylamino]-5-methoxy-benzoic acid methyl ester (Example 107, 0.028 g, 0.057 mmol) and LiOH (0.014 g, 0.57 mmol) in THF (2 mL), methanol (1 mL), and water (1 mL) was stirred at rt for 18 h and concentrated under reduced pressure. The residue was taken up in water and acidified, then filtered. The solid was dissolved in THF and purified by HPLC using waters C-18 column (55 to 90% acetonitrile/water) to give a solid (0.016 g, 58%). ¹H NMR (400 MHz, CD₃OD) δ 1.16 (t, 3H), 1.37 (t, 3H), 2.74 (q, 2H), 2.89 (q, 2H), 3.62 (s, 3H), 3.88 (s, 3H), 6.52 (d, 1H), 6.71 (d, 1H), 7.11 (t, 1H), 7.21 (d, 1H), 7.41-7.59 (m, 4H). ES-MS m/z 479.1 (MH⁺); HPLC RT (min) 3.47.

Reaction Scheme 11 summarizes the experimentals in Examples 106-108.

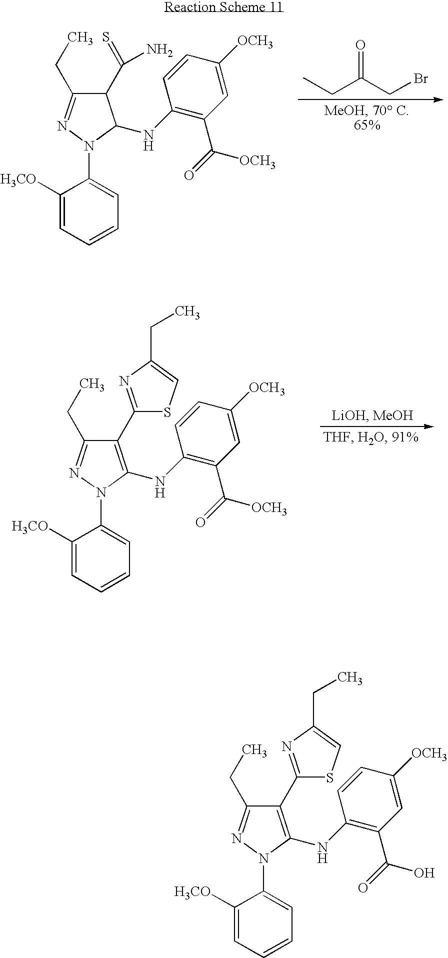

Using the methods analogous to those described above in Reaction Schemes 5, 7, 9, 10, and 11 and in Examples 98-108, and by applying the appropriate starting materials, additional compounds of the invention may also be prepared. Examples of such compounds are illustrated in Table 3 below. The specific synthetic method used to form the thiazole ring is listed next to each example.

TABLE 3

| Example | | LC-MS | |
|---|---|---|---|
| No. | Structure | Data | IUPAC Name |
| 109 | 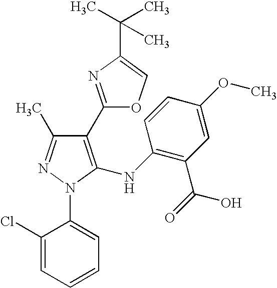 | RT = 3.85 MIN, M+ = 497.3 | 2-[4-(4-tert-Butyl-thiazol-2-yl)- 2-(2-chloro-phenyl)-5-methyl- 2H-pyrazol-3-ylamino]-5- methoxy-benzoic acid |
| 110 | 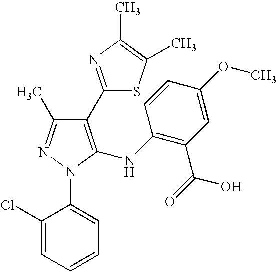 | RT = 3.21 MIN, M+ = 469.2 | 2-[2-(2-Chloro-phenyl)-4-(4,5- dimethyl-thiazol-2-yl)-5-methyl- 2H-pyrazol-3-ylamino]-5- methoxy-benzoic acid |
| 111 | 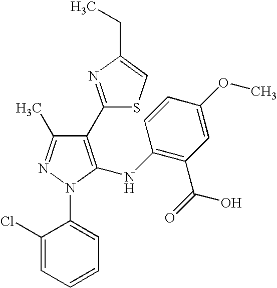 | RT = 3.41 MIN, M+ = 469.2 | 2-[2-(2-Chloro-phenyl)-4-(4- ethyl-thiazol-2-yl)-5-methyl-2H- pyrazol-3-ylamino]-5-methoxy- benzoic acid |
| 112 | 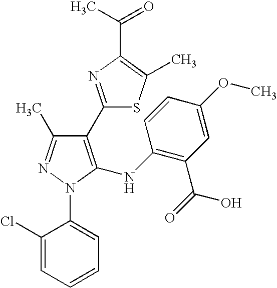 | RT = 3.30 MIN, M+ = 497.2 | 2-[4-(4-Acetyl-5-methyl-thiazol- 2-yl)-2-(2-chloro-phenyl)-5- methyl-2H-pyrazol-3-ylamino]- 5-methoxy-benzoic acid |
| 113 | 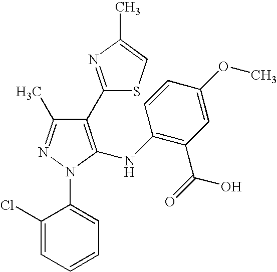 | RT = 3.22 MIN, M+ = 455.2 | 2-[2-(2-Chloro-phenyl)-5- methyl-4-(4-methyl-thiazol-2- yl)-2H-pyrazol-3-ylamino]-5- methoxy-benzoic acid |
| 114 | 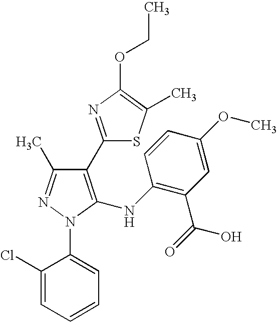 | RT = 3.69 MIN, M+ = 499.2 | 2-[2-(2-Chloro-phenyl)-4-(4- ethoxy-5-methyl-thiazol-2-yl)-5- methyl-2H-pyrazol-3-ylamino]- 5-methoxy-benzoic acid |
| 115 | 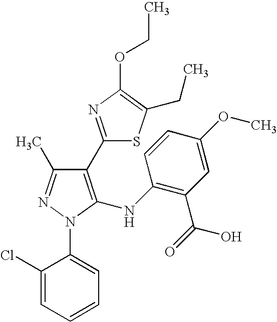 | RT = 3.85 MIN, M+ = 513.2 | 2-[2-(2-Chloro-phenyl)-4-(4- ethoxy-5-ethyl-thiazol-2-yl)-5- methyl-2H-pyrazol-3-ylamino]- 5-methoxy-benzoic acid |
| 116 | 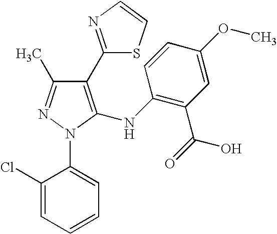 | DT = 3.07 MIN, M+ = 441.1 | 2-[2-(2-Chloro-phenyl)-5- methyl-4-thiazol-2-yl-2H- pyrazol-3-ylamino]-5-methoxy- benzoic acid |
| 117 | 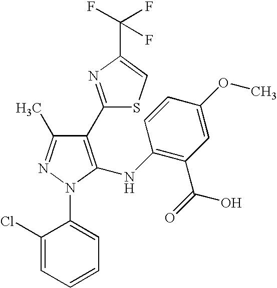 | RT = 3.62 MIN, M+ = 509.2 | 2-[2-(2-Chloro-phenyl)-5- methyl-4-(4-trifluoromethyl- thiazol-2-yl)-2H-pyrazol-3- ylamino]-5-methoxy-benzoic acid |
| 118 | 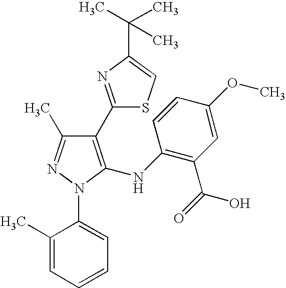 | RT = 4.07 MIN, MH+ = 477.2 | 2-[4-(4-tert-Butyl-thiazol-2-yl)- 5-methyl-2-o-tolyl-2H-pyrazol- 3-ylamino]-5-methoxy-benzoic acid |
| 119 | 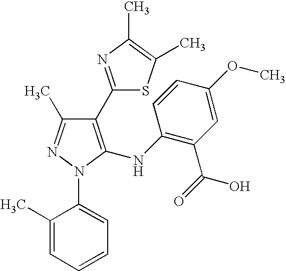 | RT = 3.37 MIN, MH+ = 449.1 | 2-[4-(4,5-Dimethyl-thiazol-2-yl)- 5-methyl-2-o-tolyl-2H-pyrazol- 3-ylamino]-5-methoxy-benzoic acid |
| 120 | 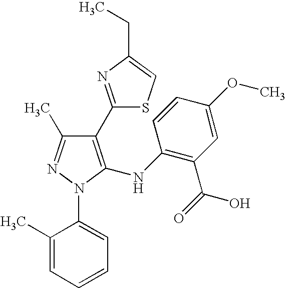 | RT 3.65 MIN, MH+ = 449.1 | 2-[4-(4-Ethyl-thiazol-2-yl)-5- methyl-2-o-tolyl-2H-pyrazol-3- ylamino]-5-methoxy-benzoic acid |
| 121 | 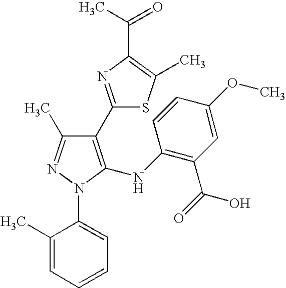 | RT 3.57 MIN, MH+ = 477.1 | 2-[4-(4-Acetyl-5-methyl-thiazol- 2-yl)-5-methyl-2-o-tolyl-2H- pyrazol-3-ylamino]-5-methoxy- benzoic acid |
| 122 | 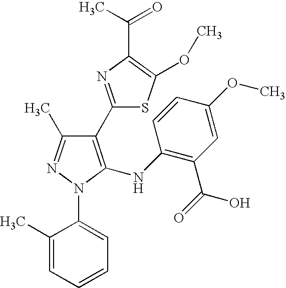 | | 2-[4-(4-Acetyl-5-methoxy- thiazol-2-yl)-5-methyl-2-o-tolyl- 2H-pyrazol-3-ylamino]-5- methoxy-benzoic acid |
| 123 | 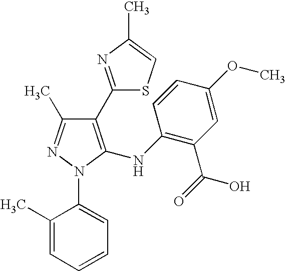 | RT 3.41 MIN, MH+ = 435.1 | 5-Methoxy-2-[5-methyl-4-(4- methyl-thiazol-2-yl)-2-o-tolyl- 2H-pyrazol-3-ylamino]-benzoic acid |
| 124 | 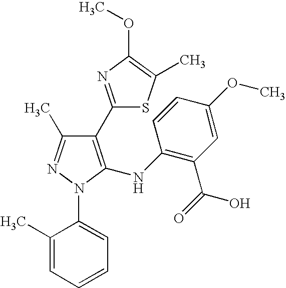 | RT = 3.86 MIN, MH+ = 465.1 | 5-Methoxy-2-[4-(4-methoxy-5- methyl-thiazol-2-yl)-5-methyl-2- o-tolyl-2H-pyrazol-3-ylamino]- benzoic acid |
| 125 | 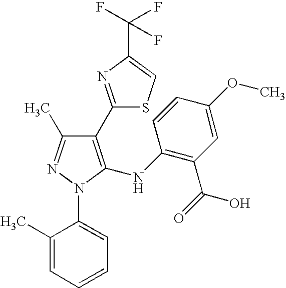 | RT = 3.96 MIN, MH+ = 489.1 | 5-Methoxy-2-[5-methyl-2-o- tolyl-4-(4-trifluoromethyl- thiazol-2-yl)-2H-pyrazol-3- ylamino]-benzoic acid |
| 126 | 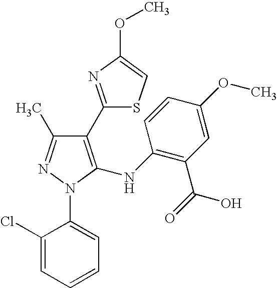 | RT = 3.19 MIN, M+ = 471.1 | 2-[2-(2-Chloro-phenyl)-4-(4- methoxy-thiazol-2-yl)-5-methyl- 2H-pyrazol-3-ylamino]-5- methoxy-benzoic acid |
| 127 | 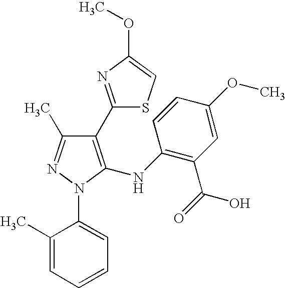 | RT = 3.18 MIN, MH+ = 451.1 | 5-Methoxy-2-[4-(4-methoxy- thiazol-2-yl)-5-methyl-2-o-tolyl- 2H-pyrazol-3-ylamino]-benzoic acid |
| 128 | 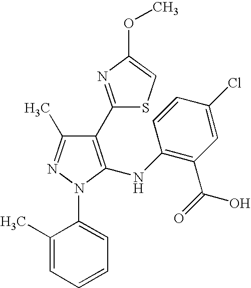 | RT = 3.48 MIN, MH+ = 455.1 | 5-Chloro-2-[4-(4-methoxy- thiazol-2-yl)-5-methyl-2-o-tolyl- 2H-pyrazol-3-ylamino]-benzoic acid |
| 129 | 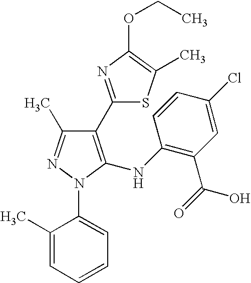 | RT = 4.00 MIN, MH+ = 483.1 | 5-Chloro-2-[4-(4-ethoxy-5- methyl-thiazol-2-yl)-5-methyl-2- o-tolyl-2H-pyrazol-3-ylamino]- benzoic acid |
| 130 | 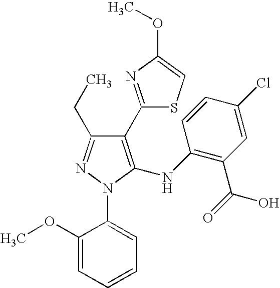 | RT = 3.53 MIN, M+ = 484.962 | 5-Chloro-2-[2-(2-methoxy- phenyl)-4-(4-methoxy-thiazol- 2-yl)-5-methyl-2H-pyrazol-3- ylamino]-benzoic acid |
| 131 | 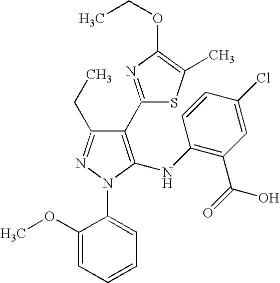 | RT = 4.05 MIN, M+ = 513.1 | 5-Chloro-2-[4-(4-ethoxy-5- methyl-thiazol-2-yl)-2-(2- methoxy-phenyl)-5-methyl-2H- pyrazol-3-ylamino]-benzoic acid |
| 132 | 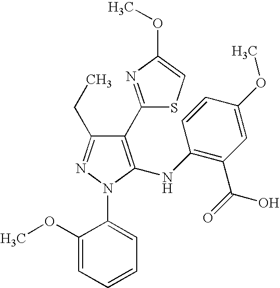 | RT = 3.25 MIN, MH+ = 481.1 | 2-[5-Ethyl-2-(2-methoxy- phenyl)-4-(4-methoxy-thiazol- 2-yl)-2H-pyrazol-3-ylamino]-5- methoxy-benzoic acid |
| 133 | 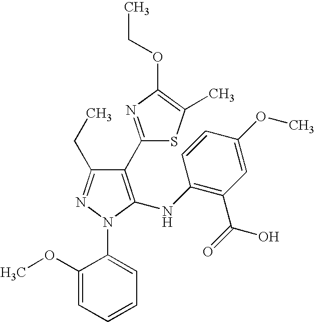 | RT = 3.72 MIN, MH+ = 509.1 | 2-[4-(4-Ethoxy-5-methyl- thiazol-2-yl)-5-ethyl-2-(2- methoxy-phenyl)-2H-pyrazol-3- ylamino]-5-methoxy-benzoic acid |
| 134 | 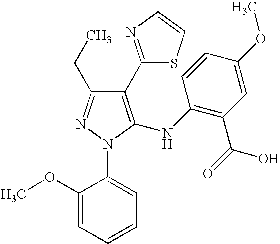 | RT = 3.12 MIN, MH+ = 451.1 | 2-[5-Ethyl-2-(2-methoxy- phenyl)-4-thiazol-2-yl-2H- pyrazol-3-ylamino]-5-methoxy- benzoic acid |
| 135 | 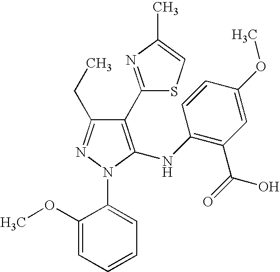 | RT = 3.25 MIN, MH+ = 465.1 | 2-[5-Ethyl-2-(2-methoxy- phenyl)-4-(4-methyl-thiazol-2- yl)-2H-pyrazol-3-ylamino]-5- methoxy-benzoic acid |
| 136 | 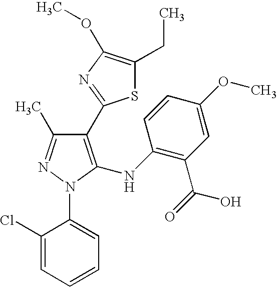 | RT = 3.73 MIN, MH+ = 499.2 | 2-[2-(2-Chloro-phenyl)-4-(5- ethyl-4-methoxy-thiazol-2-yl)-5- methyl-2H-pyrazol-3-ylamino]- 5-methoxy-benzoic acid |
| 137 | 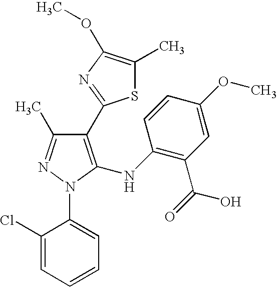 | RT = 3.59 MIN, M+ = 485.1 | 2-[2-(2-Chloro-phenyl)-4-(4- methoxy-5-methyl-thiazol-2-yl)- 5-methyl-2H-pyrazol-3- ylamino]-5-methoxy-benzoic acid |
| 138 | 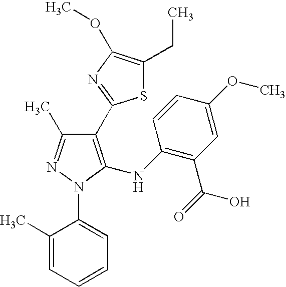 | RT = 3.75 MIN, MH+ = 479.2 | 2-[4-(5-Ethyl-4-methoxy- thiazol-2-yl)-5-methyl-2-o-tolyl- 2H-pyrazol-3-ylamino]-5- methoxy-benzoic acid |
| 139 | 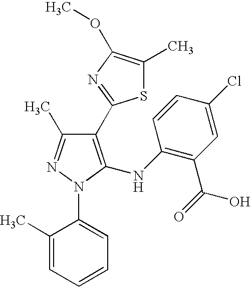 | RT = 3.90 MIN, M+ = 469.2 | 5-Chloro-2-[4-(4-methoxy-5- methyl-thiazol-2-yl)-5-methyl-2- o-tolyl-2H-pyrazol-3-ylamino]- benzoic acid |
| 140 | 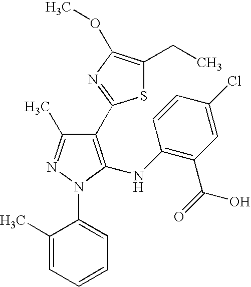 | RT = 4.04 MIN, M+ = 483.2 | 5-Chloro-2-[4-(5-ethyl-4- methoxy-thiazol-2-yl)-5-methyl- 2-o-tolyl-2H-pyrazol-3- ylamino]-benzoic acid |
| 141 | 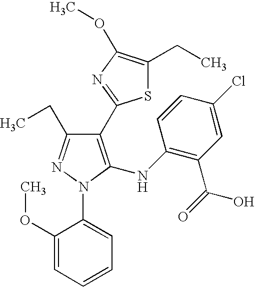 | RT = 4.08 MIN, M+ = 513.2 | 5-Chloro-2-[4-(5-ethyl-4- methoxy-thiazol-2-yl)-2-(2- methoxy-phenyl)-5-methyl-2H- pyrazol-3-ylamino]-benzoic acid |
| 142 | 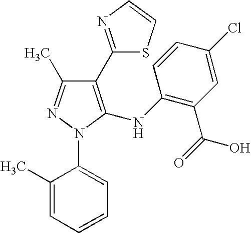 | RT = 3.32 MIN, M+ = 425.1 | 5-Chloro-2-(5-methyl-4-thiazol- 2-yl-2-o-tolyl-2H-pyrazol-3- ylamino)-benzoic acid |
| 143 | 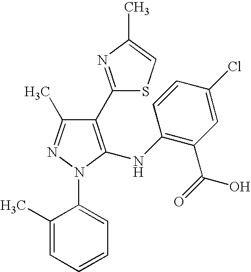 | RT = 3.49 MIN, M+ = 439.1 | 5-Chloro-2-[5-methyl-4-(4- methyl-thiazol-2-yl)-2-o-tolyl- 2H-pyrazol-3-ylamino]-benzoic acid |
| 144 | 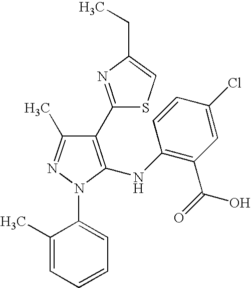 | RT = 3.72 MIN, M+ = 453.1 | 5-Chloro-2-[4-(4-ethyl-thiazol- 2-yl)-5-methyl-2-o-tolyl-2H- pyrazol-3-ylamino]-benzoic acid |
| 145 | 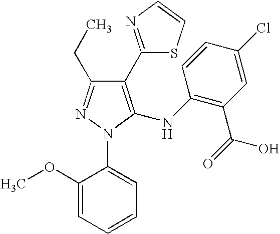 | RT = 3.42 MIN, M+ = 455.1 | 5-Chloro-2-[5-ethyl-2-(2- methoxy-phenyl)-4-thiazol-2-yl- 2H-pyrazol-3-ylamino]-benzoic acid |
| 146 | 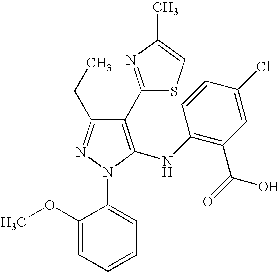 | RT = 3.58 MIN, M+ = 469.1 | 5-Chloro-2-[5-ethyl-2-(2- methoxy-phenyl)-4-(4-methyl- thiazol-2-yl)-2H-pyrazol-3- ylamino]-benzoic acid |
| 147 | 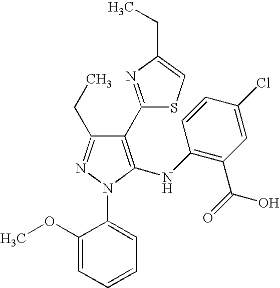 | RT = 3.80 MIN, M+ = 482.99 | 5-Chloro-2-[5-ethyl-4-(4-ethyl- thiazol-2-yl)-2-(2-methoxy- phenyl)-2H-pyrazol-3-ylamino]- benzoic acid |
| 148 | 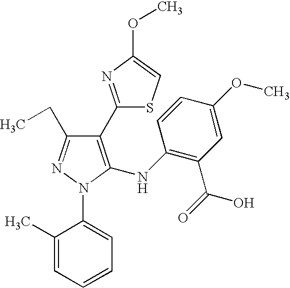 | RT = 3.37 MIN, MH+ = 465.1 | 2-[5-Ethyl-4-(4-methoxy- thiazol-2-yl)-2-o-tolyl-2H- pyrazol-3-ylamino]-5-methoxy- benzoic acid |
| 149 | 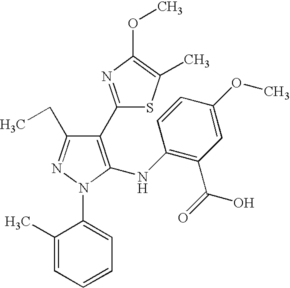 | RT = 3.72 MIN, MH+ = 479.1 | 2-[5-Ethyl-4-(4-methoxy-5- methyl-thiazol-2-yl)-2-o-tolyl- 2H-pyrazol-3-ylamino]-5- methoxy-benzoic acid |
| 150 | 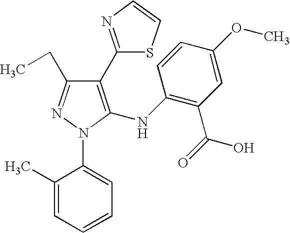 | RT = 3.25 MIN, MH+ = 435.1 | 2-(5-Ethyl-4-thiazol-2-yl-2-o- tolyl-2H-pyrazol-3-ylamino)-5- methoxy-benzoic acid |
| 151 | 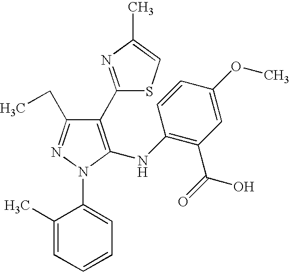 | RT = 3.38 MIN, MH+ = 449.1 | 2-[5-Ethyl-4-(4-methyl-thiazol- 2-yl)-2-o-tolyl-2H-pyrazol-3- ylamino]-5-methoxy-benzoic acid |
| 152 | 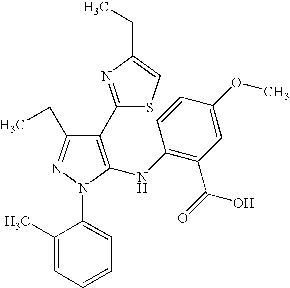 | RT = 3.61 MIN, MH+ = 463.1 | 2-[5-Ethyl-4-(4-ethyl-thiazol-2- yl)-2-o-tolyl-2H-pyrazol-3- ylamino]-5-methoxy-benzoic acid |
| 153 | 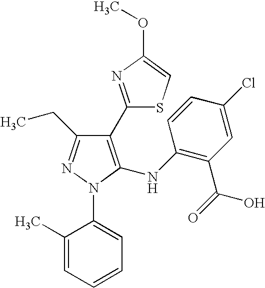 | RT = 3.65 MIN, MH+ = 469.1 | 5-Chloro-2-[5-ethyl-4-(4- methoxy-thiazol-2-yl)-2-o-tolyl- 2H-pyrazol-3-ylamino]-benzoic acid |
| 154 | 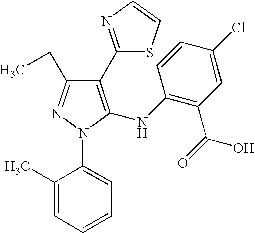 | RT = 3.54 MIN, MH+ = 439.1 | 5-Chloro-2-(5-ethyl-4-thiazol-2- yl-2-o-tolyl-2H-pyrazol-3- ylamino)-benzoic acid |
| 155 | 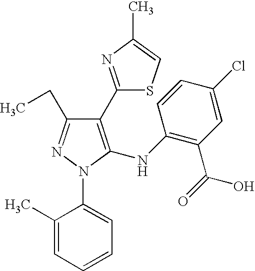 | RT = 3.72 MIN, MH+ = 453.1 | 5-Chloro-2-[5-ethyl-4-(4- methyl-thiazol-2-yl)-2-o-tolyl- 2H-pyrazol-3-ylamino]-benzoic acid |
| 156 | 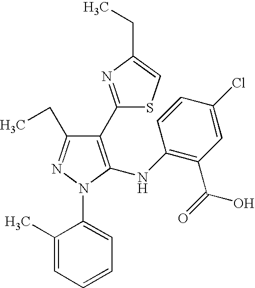 | RT = 3.93 MIN, MH+ = 467.1 | 5-Chloro-2-[5-ethyl-4-(4-ethyl- thiazol-2-yl)-2-o-tolyl-2H- pyrazol-3-ylamino]-benzoic acid |
| 157 | 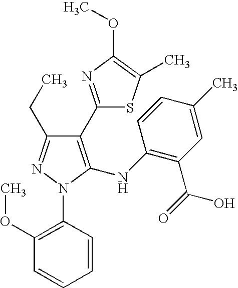 | RT = 3.79 MIN, MH+ = 479.2 | 2-[5-Ethyl-4-(4-methoxy-5- methyl-thiazol-2-yl)-2-(2- methoxy-phenyl)-2H-pyrazol-3- ylamino]-5-methyl-benzoic acid |
| 158 | 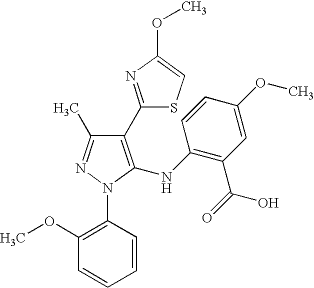 | RT = 3.06 MIN, MH+ = 467.1 | 5-Methoxy-2-[2-(2-methoxy- phenyl)-4-(4-methoxy-thiazol- 2-yl)-5-methyl-2H-pyrazol-3- ylamino]-benzoic acid |
| 159 | 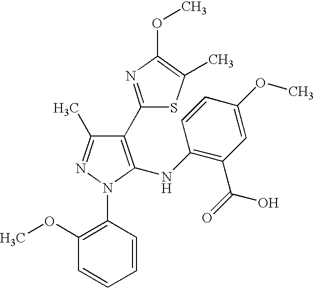 | RT = 3.39 MIN, MH+ = 481.1 | 5-Methoxy-2-[4-(4-methoxy-5- methyl-thiazol-2-yl)-2-(2- methoxy-phenyl)-5-methyl-2H- pyrazol-3-ylamino]-benzoic acid |
| 160 | 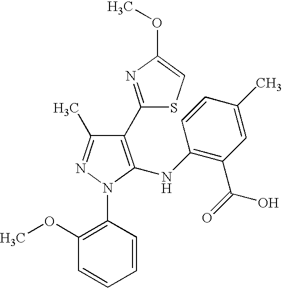 | RT = 3.22 MIN, MH+ = 451.1 | 2-[2-(2-Methoxy-phenyl)-4-(4- methoxy-thiazol-2-yl)-5-methyl- 2H-pyrazol-3-ylamino]-5- methyl-benzoic acid |
| 161 | 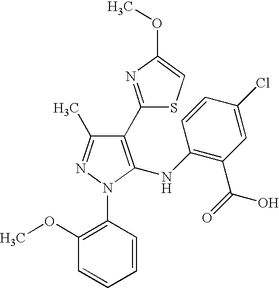 | RT = 3.35 MIN, MH+ = 471.1 | 5-Chloro-2-[2-(2-methoxy- phenyl)-4-(4-methoxy-thiazol- 2-yl)-5-methyl-2H-pyrazol-3- ylamino]-benzoic acid |
| 162 | 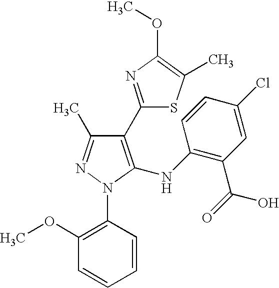 | RT = 3.70 MIN, M+ = 485.1 | 5-Chloro-2-[4-(4-methoxy-5- methyl-thiazol-2-yl)-2-(2- methoxy-phenyl)-5-methyl-2H- pyrazol-3-ylamino]-benzoic acid |
| 163 | 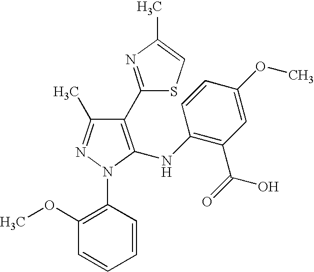 | RT = 3.05 MIN, MH+ = 451.1 | 5-Methoxy-2-[2-(2-methoxy- phenyl)-5-methyl-4-(4-methyl- thiazol-2-yl)-2H-pyrazol-3- ylamino]-benzoic acid |
| 164 | 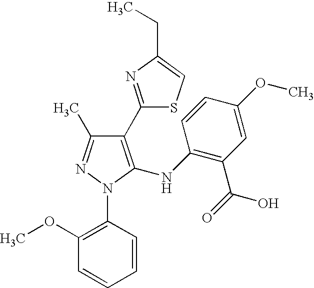 | RT = 3.23 MIN, MH+ = 465.1 | 2-[4-(4-Ethyl-thiazol-2-yl)-2-(2- methoxy-phenyl)-5-methyl-2H- pyrazol-3-ylamino]-5-methoxy- benzoic acid |
| 165 | 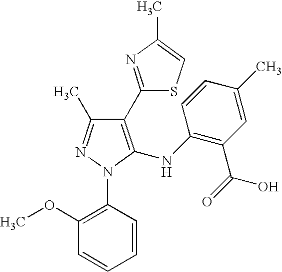 | RT = 3.21 MIN, MH+ = 435.1 | 2-[2-(2-Methoxy-phenyl)-5- methyl-4-(4-methyl-thiazol-2- yl)-2H-pyrazol-3-ylamino]-5- methyl-benzoic acid |
| 166 | 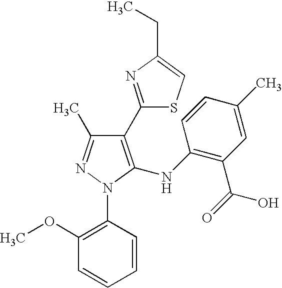 | RT = 3.43 MIN, MH+ = 449.1 | 2-[4-(4-Ethyl-thiazol-2-yl)-2-(2- methoxy-phenyl)-5-methyl-2H- pyrazol-3-ylamino]-5-methyl- benzoic acid |
| 167 | 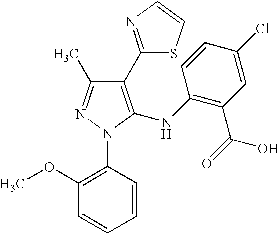 | RT = 3.23 MIN, M+ = 441.0 | 5-Chloro-2-[2-(2-methoxy- phenyl)-5-methyl-4-thiazol-2-yl- 2H-pyrazol-3-ylamino]-benzoic acid |
| 168 | 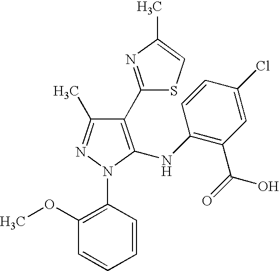 | RT = 3.38 MIN, M+ = 455.1 | 5-Chloro-2-[2-(2-methoxy- phenyl)-5-methyl-4-(4-methyl- thiazol-2-yl)-2H-pyrazol-3- ylamino]-benzoic acid |
| 169 | 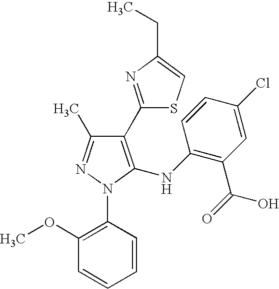 | RT = 3.60 MIN, M+ = 469.1 | 5-Chloro-2-[4-(4-ethyl-thiazol- 2-yl)-2-(2-methoxy-phenyl)-5- methyl-2H-pyrazol-3-ylamino]- benzoic acid |
| 170 | 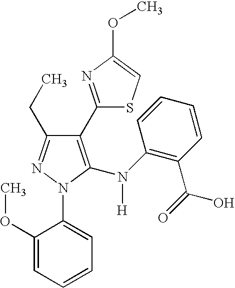 | RT = 3.33 MIN, MH+ = 451.1 | 2-[5-Ethyl-2-(2-methoxy- phenyl)-4-(4-methoxy-thiazol- 2-yl)-2H-pyrazol-3-ylamino]- benzoic acid |
| 171 | 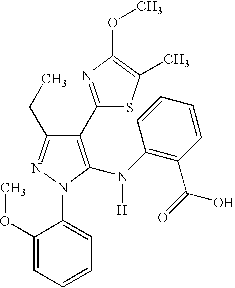 | RT = 3.68 MIN, MH+ = 465.1 | 2-[5-Ethyl-4-(4-methoxy-5- methyl-thiazol-2-yl)-2-(2- methoxy-phenyl)-2H-pyrazol-3- ylamino]-benzoic acid |
| 172 | 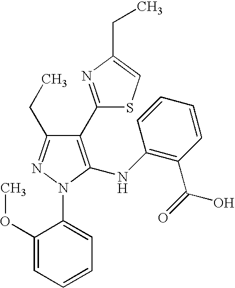 | RT = 3.57 MIN, MH+ = 449.1 | 2-[5-Ethyl-4-(4-ethyl-thiazol-2- yl)-2-(2-methoxy-phenyl)-2H- pyrazol-3-ylamino]-benzoic acid |
| 173 | 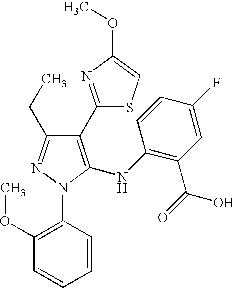 | RT = 3.36 MIN, MH+ = 469.1 | 2-[5-Ethyl-2-(2-methoxy- phenyl)-4-(4-methoxy-thiazol- 2-yl)-2H-pyrazol-3-ylamino]-5- fluoro-benzoic acid |
| 174 | 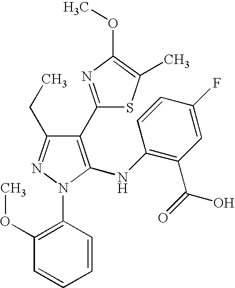 | RT = 3.70 MIN, MH+ = 483.1 | 2-[5-Ethyl-4-(4-methoxy-5- methyl-thiazol-2-yl)-2-(2- methoxy-phenyl)-2H-pyrazol-3- ylamino]-5-fluoro-benzoic acid |
| 175 | 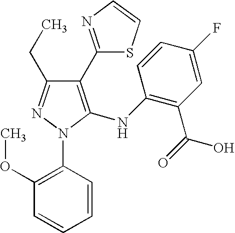 | RT = 3.21 MIN, MH+ = 439.1 | 2-[5-Ethyl-2-(2-methoxy- phenyl)-4-thiazol-2-yl-2H- pyrazol-3-ylamino]-5-fluoro- benzoic acid |
| 176 | 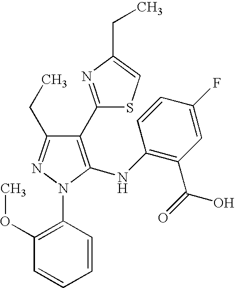 | RT = 3.60 MIN, MH+ = 467.1 | 2-[5-Ethyl-4-(4-ethyl-thiazol-2- yl)-2-(2-methoxy-phenyl)-2H- pyrazol-3-ylamino]-5-fluoro- benzoic acid |
| 177 | 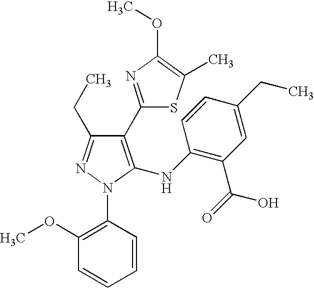 | RT = 3.64 MIN, MH+ = 493.3 | 5-Ethyl-2-[5-ethyl-4-(4- methoxy-5-methyl-thiazol-2-yl)- 2-(2-methoxy-phenyl)-2H- pyrazol-3-ylamino]-benzoic acid |
| 178 | 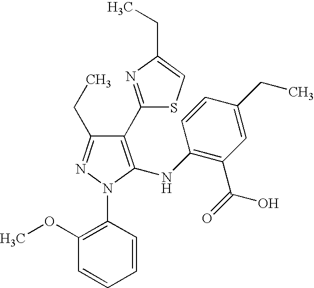 | RT = 3.61 MIN, MH+ = 477.3 | 5-Ethyl-2-[5-ethyl-4-(4-ethyl- thiazol-2-yl)-2-(2-methoxy- phenyl)-2H-pyrazol-3-ylamino]- benzoic acid |
| 179 | 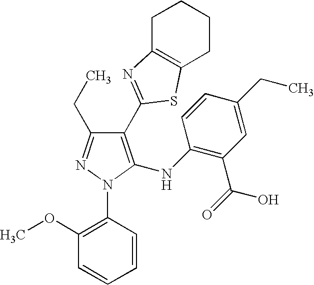 | | 5-Ethyl-2-[5-ethyl-2-(2- methoxy-phenyl)-4-(4,5,6,7- tetrahydro-benzothiazol-2-yl- 2H-pyrazol-3-ylamino]-benzoic acid |
| 180 | 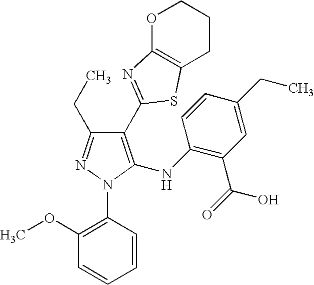 | | 2-[4-(6,7-Dihydro-5H-pyrano [2,3-d]thiazol-2-yl)-5-ethyl-2-(2- methoxy-phenyl)-2H-pyrazol-3- ylamino]-5-ethyl-benzoic acid |
| 181 | 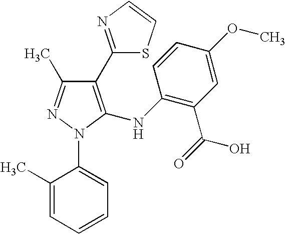 | RT = 3.36 MIN, MH+ = 421.1 | 2-(5-Methyl-4-thiazol-2-yl-2-o- tolyl-2H-pyrazol-3-ylamino)-5- methoxy-benzoic acid |

Methods of Use

As used herein, various terms are defined below.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy” or “co-therapy” means the administration of two or more therapeutic agents to treat a diabetic condition and/or disorder. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner.

The phrase “therapeutically effective” means the amount of each agent administered that will achieve the goal of improvement in a diabetic condition or disorder severity, while avoiding or minimizing adverse side effects associated with the given therapeutic treatment.

The term “pharmaceutically acceptable” means that the subject item is appropriate for use in a pharmaceutical product.

The compounds of the present invention may be employed in the treatment of diabetes, including both type 1 and type 2 diabetes (non-insulin dependent diabetes mellitus). Such treatment may also delay the onset of diabetes and diabetic complications. The compounds may be used to prevent subjects with impaired glucose tolerance from proceeding to develop type 2 diabetes. Other diseases and conditions that may be treated or prevented using compounds of the invention in methods of the invention include: Maturity-Onset Diabetes of the Young (MODY) (Herman, et al., Diabetes 43:40, 1994); Latent Autoimmune Diabetes Adult (LADA) (Zimmet, et al., Diabetes Med. 11:299, 1994); impaired glucose tolerance (IGT) (Expert Committee on Classification of Diabetes Mellitus, Diabetes Care 22 (Supp. 1):S5, 1999); impaired fasting glucose (IFG) (Charles, et al., Diabetes 40:796, 1991); gestational diabetes (Metzger, Diabetes, 40:197, 1991); and metabolic syndrome X.

The compounds of the present invention may also be effective in such disorders as obesity, and in the treatment of atherosclerotic disease, hyperlipidemia, hypercholesteremia, low HDL levels, hypertension, cardiovascular disease (including atherosclerosis, coronary heart disease, coronary artery disease, and hypertension), cerebrovascular disease and peripheral vessel disease.

The compounds of the present invention may also be useful for treating physiological disorders related to, for example, cell differentiation to produce lipid accumulating cells, regulation of insulin sensitivity and blood glucose levels, which are involved in, for example, abnormal pancreatic beta-cell function, insulin secreting tumors and/or autoimmune hypoglycemia due to autoantibodies to insulin, autoantibodies to the insulin receptor, or autoantibodies that are stimulatory to pancreatic beta-cells, macrophage differentiation which leads to the formation of atherosclerotic plaques, inflammatory response, carcinogenesis, hyperplasia, adipocyte gene expression, adipocyte differentiation, reduction in the pancreatic beta-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

Compounds of the invention may also be used in methods of the invention to treat secondary causes of diabetes (Expert Committee on Classification of Diabetes Mellitus, Diabetes Care 22 (Supp. 1 ):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenytoin, thyroid hormone, β-adrenergic agents, α-interferon and drugs used to treat HIV infection.

The compounds of the present invention may be used alone or in combination with additional therapies and/or compounds known to those skilled in the art in the treatment of diabetes and related disorders. Alternatively, the methods and compounds described herein may be used, partially or completely, in combination therapy.

The compounds of the invention may also be administered in combination with other known therapies for the treatment of diabetes, including PPAR agonists, sulfonylurea drugs, non-sulfonylurea secretagogues, α-glucosidase inhibitors, insulin sensitizers, insulin secretagogues, hepatic glucose output lowering compounds, insulin and anti-obesity drugs. Such therapies may be administered prior to, concurrently with or following administration of the compounds of the invention. Insulin includes both long and short acting forms and formulations of insulin. PPAR agonist may include agonists of any of the PPAR subunits or combinations thereof. For example, PPAR agonist may include agonists of PPAR-α, PPAR-γ, PPAR-δ or any combination of two or three of the subunits of PPAR. PPAR agonists include, for example, rosiglitazone, troglitazone, and pioglitazone. Sulfonylurea drugs include, for example, glyburide, glimepiride, chlorpropamide, tolbutamide, and glipizide. (α-glucosidase inhibitors that may be useful in treating diabetes when administered with a compound of the invention include acarbose, miglitol, and voglibose. Insulin sensitizers that may be useful in treating diabetes include PPAR-γ agonists such as the glitazones (e.g., troglitazone, pioglitazone, englitazone, MCC-555, rosiglitazone, and the like); biguanides such as metformin and phenformin; protein tyrosine phosphatase-1B (PTP-1B) inhibitors; dipeptidyl peptidase IV (DP-IV) inhibitors; and thiazolidinediones and non-thiazolidinediones. Hepatic glucose output lowering compounds that may be useful in treating diabetes when administered with a compound of the invention include metformin, such as Glucophage and Glucophage XR. Insulin secretagogues that may be useful in treating diabetes when administered with a compound of the invention include sulfonylurea and non-sulfonylurea drugs: GLP-1, GIP, secretin, nateglinide, meglitinide, repaglinide, glibenclamide, glimepiride, chlorpropamide, glipizide. GLP-1 includes derivatives of GLP-1 with longer half-lives than native GLP-1, such as, for example, fatty-acid derivatized GLP-1 and exendin. In one embodiment of the invention, compounds of the invention are used in combination with insulin secretagogues to increase the sensitivity of pancreatic β-cells to the insulin secretagogue.

Compounds of the invention may also be used in methods of the invention in combination with anti-obesity drugs. Anti-obesity drugs include β-3 adrenergic receptor agonists; CB-1 (cannabinoid) receptor antagonists; neuropeptide Y antagonists; appetite suppressants, such as, for example, sibutramine (Meridia); and lipase inhibitors, such as, for example, orlistat (Xenical). Compounds of the present invention may be administered in combination with other pharmaceutical agents, such as apo-B/MTP inhibitors, MCR-4 agonists, CCK-A agonists, monoamine reuptake inhibitors, sympathomimetic agents, dopamine agonists, melanocyte-stimulating hormone receptor analogs, melanin concentrating hormone antagonists, leptins, leptin analogs, leptin receptor agonists, galanin antagonists, lipase inhibitors, bombesin agonists, thyromimetic agents, dehydroepiandrosterone or analogs thereof, glucocorticoid receptor agonists or antagonists, orexin receptor antagonists, urocortin binding protein antagonists, ciliary neurotrophic factors, AGRPs (human agouti-related proteins), ghrelin receptor antagonists, histamine 3 receptor antagonists or reverse agonists, neuromedin U receptor agonists, and the like.

Compounds of the invention may also be used in methods of the invention in combination with drugs commonly used to treat lipid disorders in diabetic patients. Such drugs include, but are not limited to, HMG-CoA reductase inhibitors, nicotinic acid, lipid lowering drugs (e.g., stanol esters, sterol glycosides such as tiqueside, and azetidinones such as ezetimibe), ACAT inhibitors (such as avasimibe), bile acid sequestrants, bile acid reuptake inhibitors, microsomal triglyceride transport inhibitors, and fibric acid derivatives. HMG-CoA reductase inhibitors include, for example, lovastatin, simvastatin, pravastatin, fluvastatin, atorvastatin, rivastatin, itavastatin, cerivastatin, and ZD-4522. Fibric acid derivatives include, for example, clofibrate, fenofibrate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Compounds of the invention may also be used in combination with anti-hypertensive drugs, such as, for example, β-blockers and ACE inhibitors. Examples of additional anti-hypertensive agents for use in combination with the compounds of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e. g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

Such co-therapies may be administered in any combination of two or more drugs (e.g., a compound of the invention in combination with an insulin sensitizer and an anti-obesity drug). Such co-therapies may be administered in the form of pharmaceutical compositions, as described above.

Based on well known assays used to determine the efficacy for treatment of conditions identified above in mammals, and by comparison of these results with the results of known medicaments that are used to treat these conditions, the effective dosage of the compounds of this invention can readily be determined for treatment of each desired indication. The amount of the active ingredient (e.g., compounds) to be administered in the treatment of one of these conditions can vary widely according to such considerations as the particular compound and dosage unit employed, the mode of administration, the period of treatment, the age and sex of the patient treated, and the nature and extent of the condition treated,

The total amount of the active ingredient to be administered may generally range from about 0.0001 mg/kg to about 200 mg/kg, and preferably from about 0.01 mg/kg to about 200 mg/kg body weight per day. A unit dosage may contain from about 0.05 mg to about 1500 mg of active ingredient, and may be administered one or more times per day. The daily dosage for administration by injection, including intravenous, intramuscular, subcutaneous, and parenteral injections, and use of infusion techniques may be from about 0.01 to about 200 mg/kg. The daily rectal dosage regimen may be from 0.01 to 200 mg/kg of total body weight. The transdermal concentration may be that required to maintain a daily dose of from 0.01 to 200 mg/kg.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present invention may be ascertained by those skilled in the art using conventional treatment tests.

The compounds of this invention may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which are comprised of a pharmaceutically acceptable carrier and a therapeutically effective amount of a compound. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a compound is that amount which produces a result or exerts an influence on the particular condition being treated. The compounds described herein may be administered with a pharmaceutically-acceptable carrier using any effective conventional dosage unit forms, including, for example, immediate and timed release preparations, orally, parenterally, topically, or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this invention may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug (e.g., compound) with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

Another formulation employs the use of biodegradable microspheres that allow controlled, sustained release of pharmaceutical agents. Such formulations can be comprised of synthetic polymers or copolymers. Such formulations allow for injection, inhalation, nasal, or oral administration. The construction and use of biodegradable microspheres for the delivery of pharmaceutical agents is well known in the art (e.g., U.S. Pat. No. 6,706,289, incorporated herein by reference).

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds described herein may be administered as the sole pharmaceutical agent or in combination with one or more other pharmaceutical agents where the combination causes no unacceptable adverse effects. For example, the compounds of this invention can be combined with known anti-obesity, or with known antidiabetic or other indication agents, and the like, as well as with admixtures and combinations thereof.

The compounds described herein may also be utilized, in free base form or in compositions, in research and diagnostics, or as analytical reference standards, and the like. Therefore, the present invention includes compositions which are comprised of an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000).

It should be apparent to one of ordinary skill in the art that changes and modifications can be made to this invention without departing from the spirit or scope of the invention as it is set forth herein.

### Biological Evaluation

In order that this invention may be better understood, the following examples are set forth. These examples are for the purpose of illustration only, and are not to be construed as limiting the scope of the invention in any manner. All publications mentioned herein are incorporated by reference in their entirety.

Demonstration of the activity of the compounds of the present invention may be accomplished through in vitro, ex vivo, and in vivo assays that are well known in the art. For example, to demonstrate the efficacy of a pharmaceutical agent for the treatment of diabetes and related disorders such as Syndrome X, impaired glucose tolerance, impaired fasting glucose, and hyperinsulinemia, the following assays may be used.

In vitro Assay

Insulin Secretion from INS-1 Cells

INS-1 cells were isolated from X-ray induced rat insulinoma (Asfari, et al., Endocrinology 130:167, 1992). INS-1 cells were seeded at 30,000 cells per well in Biocoat Collagen1 Cellware 96-well plates and incubated for 4-5 days. The cells were then treated for 2 days with complete media (RPMI 1640, 10% Fetal Bovine Serum, 100 μg/mL Penicillin/Streptomycin, 0.5 mM sodium pyruvate, 10 mM HEPES, and 50 μM beta-mercaptoethanol) adjusted to 3 mM glucose. After the two-day treatment, the cells were washed with Krebs-Ringer-Bicarbonate-HEPES (KRBH) containing 3 mM glucose. The cells were then incubated for 30 min in the same buffer. The cells were incubated for an additional 2 h in the presence of the desired concentration of glucose and compounds. The supernatants were harvested.

To determine the amount of insulin secreted, the supernatants were mixed with anti-insulin antibody and a tracer amount of ¹²⁵I-insulin in phosphate buffered saline containing 0.5% bovine serum albumin. Protein A coated SPA (scintillation proximity assay) beads were added. The plates were incubated for 5-20 h and counted on a scintillation counter to measure insulin levels. Activity for compounds at a given concentration was expressed as a fold-stimulation of insulin secretion relative to controls.

Insulin Secretion from Dispersed Rat Islet Cells

Insulin secretion of dispersed rat islets mediated by a number of compounds of the present invention was measured as follows. Islets of Langerhans, isolated from male Sprague-Dawley rats (200-250 g), were digested using collagenase. The dispersed islet cells were treated with trypsin, seeded into 96 V-bottom plates, and pelleted. The cells were then cultured overnight in media with or without compounds of this invention. The media was aspirated, and the cells were pre-incubated with Krebs-Ringer-HEPES buffer containing 3 mM glucose for 30 minutes at 37° C. The pre-incubation buffer was removed, and the cells were incubated at 37° C. with Krebs-Ringer-HEPES buffer containing the appropriate glucose concentration (e.g., 8 mM) with or without compounds for an appropriate time. In some studies, an appropriate concentration of GLP-1 or forskolin was also included. A portion of the supernatant was removed and its insulin content was measured by SPA. The results were expressed as “fold over control” (FOC).

In vivo Assay

Effect of Compounds on Intraperitoneal Glucose Tolerance in Rats

The in vivo activities of the compounds of this invention when administered via oral gavage were examined in rats. Rats fasted overnight were given an oral dose of vehicle control or compound. Three hours later, basal blood glucose was measured, and the rats were given 2 g/kg of glucose intraperitoneally. Blood glucose was measured again after 15, 30, and 60 min. The representative compounds of this invention significantly reduced blood glucose levels relative to the vehicle following the IPGTT (Intraperitoneal Glucose Tolerance Test).

Method for Measuring an Effect on Cardiovascular Parameters

Cardiovascular parameters (e.g., heart rate and blood pressure) are also evaluated. SHR rats are orally dosed once daily with vehicle or test compound for 2 weeks. Blood pressure and heart rate are determined using a tail-cuff method as described by Grinsell, et al., (Am. J. Hypertens. 13:370-375, 2000). In monkeys, blood pressure and heart rate are monitored as described by Shen, et al., (J. Pharmacol. Exp. Therap. 278:1435-1443, 1996).

Method for Measuring Triglyceride Levels

hApoA1 mice (obtained from Jackson Laboratories, Bar Harbor, Me.) are bled (by either eye or tail vein) and grouped according to equivalent mean serum triglyceride levels. They are dosed orally (by gavage in a pharmaceutically acceptable vehicle) with the test compound once daily for 8 days. The animals are then bled again by eye or tail vein, and serum triglyceride levels are determined. In each case, triglyceride levels are measured using a Technicon Axon Autoanalyzer (Bayer Corporation, Tarrytown, N.Y.).

Method for Measuring HDL-Cholesterol Levels

To determine plasma HDL-cholesterol levels, hApoA1 mice are bled and grouped with equivalent mean plasma HDL-cholesterol levels. The mice are orally dosed once daily with vehicle or test compound for 7 days, and then bled again on day 8. Plasma is analyzed for HDL-cholesterol using the Synchron Clinical System (CX4) (Beckman Coulter, Fullerton, Calif.).

Method for Measuring Total Cholesterol, HDL-Cholesterol, Triglycerides, and Glucose Levels

In another in vivo assay, obese monkeys are bled, then orally dosed once daily with vehicle or test compound for 4 weeks, and then bled again. Serum is analyzed for total cholesterol, HDL-cholesterol, triglycerides, and glucose using the Synchron Clinical System (CX4) (Beckman Coulter, Fullerton, Calif.). Lipoprotein subclass analysis is performed by NMR spectroscopy as described by Oliver, et al., (Proc. Natl. Acad. Sci. USA 98:5306-5311, 2001).

All publications and patents mentioned in the above specification are incorporated herein by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A compound of Formula (I) 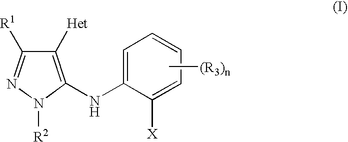
wherein
R¹ is H,
(C₁-C₆)alkyl optionally substituted with one substituent selected from the group consisting of (C₁-C₄)alkoxy, phenyl optionally substituted with halo, and [tri(C₁-C₄)alkyl]silyl,
(C₃-C₆)alkenyl,
(C₃-C₆)alkynyl,
(C₃-C₆)cycloalkyl optionally substituted with up to two substituents selected from the group consisting of (C₁-C₃)alkyl, CF₃, and halo,
(C₁-C₆)haloalkyl, or
phenyl optionally substituted with up to four substituents selected from the group consisting of
halo,
(C₁-C₆)alkyl optionally substituted with one (C₁-C₄)alkoxy or oxo,
(C₁-C₆)alkoxy,
(C₁-C₃)haloalkyl,
(C₁-C₃)haloalkoxy,
(C₃-C₆)cycloalkyl,
NR⁴R⁴,
cyano, and
(C₁-C₆)alkylthio;
Het is a mono heterocyclic ring radical selected from the group consisting of thienyl, furyl, oxazolyl, isoxazolyl, imidazolyl, thiazolyl, isothiazolyl, pyrrolyl, pyrazolyl, and thiadiazolyl,
each of which may be optionally substituted with up to two substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo,
or
optionally fused to a 5- or 6-membered saturated or partially saturated carbocyclic ring or to a 5- or 6-membered saturated or unsaturated heterocyclic ring containing 1-3 heteroatoms selected from N, O, and S,
or
is a bicyclic heterocylic ring radical selected from the group consisting of 2-benzothienyl, 3-benzothienyl, 2-benzofuryl, 3-benzofuryl, 2-benzoazolyl, and 2-benzothiazolyl
each of which may be optionally substituted with up to four substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo;
R² is (C₁-C₆)alkyl,
(C₃-C₆)cycloalkyl,
(C₂-C₃)haloalkyl,
benzyl optionally substituted on the aryl ring with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl,
or
phenyl optionally substituted with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl;
R³ is (C₁-C₆)alkyl optionally substituted with one (C₁-C₄)alkoxy or oxo,
(C₁-C₆)alkoxy,
(C₁-C₆)alkylthio,
(C₁-C₃)haloalkyl,
(C₁-C₃)haloalkoxy,
halo, or
NR⁴R⁴;
n=0, 1, 2, or 3;
X is CO₂R⁴;
R⁴ is H,
(C₁-C₆)alkyl,
benzyl optionally substituted on the aryl ring with up to four substituents selected from the group consisting of
halo,
(C₁-C₆)alkyl optionally substituted with one (C₁-C₄)alkoxy,
(C₁-C₃)alkoxy,
(C₁-C₃)haloalkyl,
(C₁-C₃)haloalkoxy,
cyano, and
(C₁-C₆)alkylthio,
or
phenyl optionally substituted with up to four substituents selected from the group consisting of
(C₁-C₆)alkyl optionally substituted with one (C₁-C₄)alkoxy,
halo,
(C₁-C₆)alkoxy,
(C₁-C₃)haloalkyl,
(C₁-C₃)haloalkoxy,
cyano, and
(C₁-C₆)alkylthio;
or the pharmaceutically acceptable salts thereof.

2. The compound of claim 1, wherein
R¹ is H,
(C₁-C₆)alkyl optionally substituted with one substituent selected from the group consisting of (C₁-C₄)alkoxy, phenyl optionally substituted with halo, and [tri(C₁-C₄)alkyl]silyl,
(C₃-C₆)alkenyl,
(C₃-C₆)alkynyl,
(C₃-C₆)cycloalkyl optionally substituted with up to two substituents selected from the group consisting of (C₁-C₃)alkyl, CF₃, and halo,
(C₁-C₆)haloalkyl, or
phenyl optionally substituted with up to four substituents selected from the group consisting of
halo,
(C₁-C₆)alkyl optionally substituted with one (C₁-C₄)alkoxy or oxo,
(C₁-C₆)alkoxy,
(C₁-C₃)haloalkyl,
(C₁-C₃)haloalkoxy,
(C₃-C₆)cycloalkyl,
NR⁴R⁴,
cyano, and
(C₁-C₆)alkylthio;
Het is a mono heterocyclic ring radical selected from the group consisting of thienyl, furyl, oxazolyl, isoxazolyl, imidazolyl, thiazolyl, isothiazolyl, pyrrolyl, pyrazolyl, and thiadiazolyl,
each of which may be optionally substituted with up to two substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo,
or
optionally fused to a 5- or 6-membered saturated or partially saturated carbocyclic ring or to a 5- or 6-membered saturated or unsaturated heterocyclic ring containing 1-3 heteroatoms selected from N, O, and S;
R² is (C₁-C₆)alkyl,
(C₃-C₆)cycloalkyl,
(C₂-C₃)haloalkyl,
benzyl optionally substituted on the aryl ring with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl,
or
phenyl optionally substituted with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl;
R³ is (C₁-C₆)alkyl optionally substituted with one (C₁-C₄)alkoxy or oxo,
(C₁-C₆)alkoxy,
(C₁-C₆)alkylthio,
(C₁-C₃)haloalkyl,
(C₁-C₃)haloalkoxy,
halo, or
NR⁴R⁴;
n=0, 1, 2, or 3;
X is CO₂R⁴;
R⁴ is H,
(C₁-C₆)alkyl,
benzyl optionally substituted on the aryl ring with up to four substituents selected from the group consisting of
halo,
(C₁-C₆)alkyl optionally substituted with one (C₁-C₄)alkoxy,
(C₁-C₃)alkoxy,
(C₁-C₃)haloalkyl,
(C₁-C₃)haloalkoxy,
cyano, and
(C₁-C₆)alkylthio,
or
phenyl optionally substituted with up to four substituents selected from the group consisting of
(C₁-C₆)alkyl optionally substituted with one (C₁-C₄)alkoxy,
halo,
(C₁-C₆)alkoxy,
(C₁-C₃)haloalkyl,
(C₁-C₃)haloalkoxy,
cyano, and
(C₁-C₆)alkylthio.

3. The compound of claim 1, wherein
R¹ is H,
(C₁-C₆)alkyl optionally substituted with one substituent selected from the group consisting of (C₁-C₄)alkoxy, phenyl optionally substituted with halo, and [tri(C₁-C₄)alkyl]silyl,
(C₃-C₆)alkenyl,
(C₃-C₆)alkynyl,
(C₃-C₆)cycloalkyl optionally substituted with up to two substituents selected from the group consisting of (C₁-C₃)alkyl, CF₃, and halo,
(C₁-C₆)haloalkyl, or
phenyl optionally substituted with up to four substituents selected from the group consisting of
halo,
(C₁-C₆)alkyl optionally substituted with one (C₁-C₄)alkoxy or oxo,
(C₁-C₆)alkoxy,
(C₁-C₃)haloalkyl,
(C₁-C₃)haloalkoxy,
(C₃-C₆)cycloalkyl,
NR⁴R⁴,
cyano, and
(C₁-C₆)alkylthio;
Het is a bicyclic heterocylic ring radical selected from the group consisting of 2-benzothienyl, 3-benzothienyl, 2-benzofuryl, 3-benzofuryl, 2-benzoazolyl, and 2-benzothiazolyl
each of which may be optionally substituted with up to four substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo;
R² is (C₁-C₆)alkyl,
(C₃-C₆)cycloalkyl,
(C₂-C₃)haloalkyl,
benzyl optionally substituted on the aryl ring with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl,
or
phenyl optionally substituted with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl;
R³ is (C₁-C₆)alkyl optionally substituted with one (C₁-C₄)alkoxy or oxo,
(C₁-C₆)alkoxy,
(C₁-C₆)alkylthio,
(C₁-C₃)haloalkyl,
(C₁-C₃)haloalkoxy,
halo, or
NR⁴R⁴;
n=0, 1, 2, or 3;
X is CO₂R⁴;
R⁴ is H,
(C₁-C₆)alkyl,
benzyl optionally substituted on the aryl ring with up to four substituents selected from the group consisting of
halo,
(C₁-C₆)alkyl optionally substituted with one (C₁-C₄)alkoxy,
(C₁-C₃)alkoxy,
(C₁-C₃)haloalkyl,
(C₁-C₃)haloalkoxy,
cyano, and
(C₁-C₆)alkylthio,
or
phenyl optionally substituted with up to four substituents selected from the group consisting of
(C₁-C₆)alkyl optionally substituted with one (C₁-C₄)alkoxy,
halo,
(C₁-C₆)alkoxy,
(C₁-C₃)haloalkyl,
(C₁-C₃)haloalkoxy,
cyano, and
(C₁-C₆)alkylthio.

4. The compound of claim 1, wherein
R¹ is H,
(C₁-C₆)alkyl optionally substituted with one substituent selected from the group consisting of (C₁-C₄)alkoxy, phenyl optionally substituted with halo, and [tri(C₁-C₄)alkyl]silyl,
(C₃-C₆)alkenyl,
(C₃-C₆)alkynyl, or
(C₁-C₆)haloalkyl;
Het is a mono heterocyclic ring radical selected from the group consisting of thienyl, furyl, oxazolyl, isoxazolyl, imidazolyl, thiazolyl, isothiazolyl, pyrrolyl, pyrazolyl, and thiadiazolyl,
each of which may be optionally substituted with up to two substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo,
or
optionally fused to a 5- or 6-membered saturated or partially saturated carbocyclic ring or to a 5- or 6-membered saturated or unsaturated heterocyclic ring containing 1-3 heteroatoms selected from N, O, and S,
or
is a bicyclic heterocylic ring radical selected from the group consisting of 2-benzothienyl, 3-benzothienyl, 2-benzofuryl, 3-benzofuryl, 2-benzoazolyl, and 2-benzothiazolyl
each of which may be optionally substituted with up to four substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo;
R² is (C₁-C₆)alkyl,
(C₃-C₆)cycloalkyl,
(C₂-C₃)haloalkyl,
benzyl optionally substituted on the aryl ring with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl,
or
phenyl optionally substituted with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl;
R³ is (C₁-C₆)alkyl optionally substituted with one (C₁-C₄)alkoxy or oxo,
(C₁-C₆)alkoxy,
(C₁-C₆)alkylthio,
(C₁-C₃)haloalkyl,
(C₁-C₃)haloalkoxy, or
halo;
n=0, 1, 2, or 3;
X is CO₂R⁴;
R⁴ is H or (C₁-C₆)alkyl

5. The compound of claim 1, wherein
R¹ is H,
(C₁-C₆)alkyl optionally substituted with one substituent selected from the group consisting of (C₁-C₄)alkoxy, phenyl optionally substituted with halo, and [tri(C₁-C₄)alkyl]silyl,
(C₃-C₆)alkenyl,
(C₃-C₆)alkynyl, or
(C₁-C₆)haloalkyl;
Het is a mono heterocyclic ring radical selected from the group consisting of thienyl, furyl, oxazolyl, isoxazolyl, imidazolyl, thiazolyl, isothiazolyl, pyrrolyl, pyrazolyl, and thiadiazolyl,
each of which may be optionally substituted with up to two substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo,
or
optionally fused to a 5- or 6-membered saturated or partially saturated carbocyclic ring or to a 5- or 6-membered saturated or unsaturated heterocyclic ring containing 1-3 heteroatoms selected from N, O, and S;
R² is
phenyl optionally substituted with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl;
R³ is (C₁-C₆)alkyl optionally substituted with one (C₁-C₄)alkoxy or oxo,
(C₁-C₆)alkoxy,
(C₁-C₃)haloalkyl,
(C₁-C₃)haloalkoxy, or
halo;
n=0, 1, 2, or 3;
X is CO₂R⁴;
R⁴ is H or (C₁-C₆)alkyl.

6. The compound of claim 1, wherein
R¹ is
(C₃-C₆)cycloalkyl optionally substituted with up to two substituents selected from the group consisting of (C₁-C₃)alkyl, CF₃, and halo,
or
phenyl optionally substituted with up to four substituents selected from the group consisting of
halo,
(C₁-C₆)alkyl optionally substituted with one (C₁-C₄)alkoxy or oxo,
(C₁-C₆)alkoxy,
(C₁-C₃)haloalkyl,
(C₁-C₃)haloalkoxy,
(C₃-C₆)cycloalkyl,
NR⁴R⁴,
cyano, and
(C₁-C₆)alkylthio;
Het is a mono heterocyclic ring radical selected from the group consisting of thienyl, furyl, oxazolyl, isoxazolyl, imidazolyl, thiazolyl, isothiazolyl, pyrrolyl, pyrazolyl, and thiadiazolyl,
each of which may be optionally substituted with up to two substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo,
or
optionally fused to a 5- or 6-membered saturated or partially saturated carbocyclic ring or to a 5- or 6-membered saturated or unsaturated heterocyclic ring containing 1-3 heteroatoms selected from N, O, and S;
R² is
phenyl optionally substituted with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl;
R³ is (C₁-C₆)alkyl optionally substituted with one (C₁-C₄)alkoxy or oxo,
(C₁-C₆)alkoxy,
(C₁-C₃)haloalkyl,
(C₁-C₃)haloalkoxy,
halo;
n=0, 1, 2, or 3;
X is CO₂R⁴;
R⁴ is H or (C₁-C₆)alkyl.

7. The compound of claim 1, wherein
R¹ is H,
(C₁-C₆)alkyl optionally substituted with one substituent selected from the group consisting of (C₁-C₄)alkoxy, phenyl optionally substituted with halo, and [tri(C₁-C₄)alkyl]silyl,
(C₃-C₆)alkenyl,
(C₃-C₆)alkynyl,
(C₃-C₆)cycloalkyl optionally substituted with up to two substituents selected from the group consisting of (C₁-C₃)alkyl, CF₃, and halo,
or
(C₁-C₆)haloalkyl;
Het is a bicyclic heterocylic ring radical selected from the group consisting of 2-benzothienyl, 3-benzothienyl, 2-benzofuryl, 3-benzofuryl, 2-benzoazolyl, and 2-benzothiazolyl
each of which may be optionally substituted with up to four substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo;
R² is (C₁-C₆)alkyl,
(C₃-C₆)cycloalkyl,
(C₂-C₃)haloalkyl,
benzyl optionally substituted on the aryl ring with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl,
or
phenyl optionally substituted with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl;
R³ is (C₁-C₆)alkyl optionally substituted with one (C₁-C₄)alkoxy or oxo,
(C₁-C₆)alkoxy,
(C₁-C₆)alkylthio,
(C₁-C₃)haloalkyl,
(C₁-C₃)haloalkoxy,
halo, or
NR⁴R⁴;
n=0, 1, 2, or 3;
X is CO₂R⁴;
R⁴ is H,
(C₁-C₆)alkyl,
benzyl optionally substituted on the aryl ring with up to four substituents selected from the group consisting of
halo,
(C₁-C₆)alkyl optionally substituted with one (C₁-C₄)alkoxy,
(C₁-C₃)alkoxy,
(C₁-C₃)haloalkyl,
(C₁-C₃)haloalkoxy,
cyano, and
(C₁-C₆)alkylthio,
or
phenyl optionally substituted with up to four substituents selected from the group consisting of
(C₁-C₆)alkyl optionally substituted with one (C₁-C₄)alkoxy,
halo,
(C₁-C₆)alkoxy,
(C₁-C₃)haloalkyl,
(C₁-C₃)haloalkoxy,
cyano, and
(C₁-C₆)alkylthio.

8. The compound of claim 1, wherein
R¹ is H,
(C₁-C₆)alkyl optionally substituted with one substituent selected from the group consisting of (C₁-C₄)alkoxy, phenyl optionally substituted with halo, and [tri(C₁-C₄)alkyl]silyl,
(C₃-C₆)alkenyl,
(C₃-C₆)alkynyl, or
(C₁-C₆)haloalkyl;
Het is a mono heterocyclic ring radical selected from the group consisting of thienyl, furyl, oxazolyl, isoxazolyl, imidazolyl, thiazolyl, isothiazolyl, pyrrolyl, pyrazolyl, and thiadiazolyl,
each of which may be optionally substituted with up to two substituents selected from the group consisting of (C₁-C₆)alkoxy, (C₁-C₆)haloalkyl, (C₁-C₆)alkylthio, halo, cyano, and (C₁-C₆)alkyl optionally substituted with one (C₁-C₄)alkoxy or oxo;
R² is (C₁-C₆)alkyl,
(C₃-C₆)cycloalkyl,
(C₂-C₃)haloalkyl,
benzyl optionally substituted on the aryl ring with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl,
or
phenyl optionally substituted with up to four substituents selected from the group consisting of
(C₁-C₆)alkyl optionally substituted with one (C₁-C₄)alkoxy or oxo,
halo,
(C₁-C₃)haloalkyl,
(C₁-C₆)alkoxy,
(C₁-C₃)haloalkoxy,
NR⁴R⁴,
cyano,
(C₁-C₆)alkylthio, and
SO₂(C₁-C₃)alkyl;
R³ is (C₁-C₆)alkyl optionally substituted with one (C₁-C₄)alkoxy or oxo,
(C₁-C₆)alkoxy,
(C₁-C₃)haloalkyl,
(C₁-C₃)haloalkoxy, or
halo;
n=0, 1,or2;
X is CO₂R⁴;
R⁴ is H, or (C₁-C₆)alkyl.

9. The compound of claim 1 selected from the group consisting of:
2-[2-(2-chloro-phenyl)-4-(4-ethoxy-5-methyl-thiazol-2-yl)-5-methyl-2H-pyrazol-3-ylamino]-5-methoxy-benzoic acid;
2-[4-(4,5-cimethyl-thiazol-2-yl)-5-methyl-2-o-tolyl-2H-pyrazol-3-ylamino]-5-methoxy-benzoic acid;
5-methoxy-2-[4-(4-methoxy-5-methyl-thiazol-2-yl)-5-methyl-2-o-tolyl-2H-pyrazol-3-ylamino]-benzoic acid;
2-(5-methyl-4-thiazol-2-yl-2-o-tolyl-2H-pyrazol-3-ylamino)-5-methoxy-benzoic acid;
2-[4-(4-ethyl-oxazol-2-yl)-5-methyl-2-o-tolyl-2H-pyrazol-3-ylamino]-5-methoxy-benzoic acid;
2-[2-(2-chloro-phenyl)-4-(4-methoxy-thiazol-2-yl)-5-methyl-2H-pyrazol-3-ylamino]-5-methoxy-benzoic acid;
5-methoxy-2-[4-(4-methoxy-thiazol-2-yl)-5-methyl-2-o-tolyl-2H-pyrazol-3-ylamino]-benzoic acid;
5-chloro-2-[2-(2-methoxy-phenyl)-4-(4-methoxy-thiazol-2-yl)-5-methyl-2H-pyrazol-3-ylamino]-benzoic acid;
2-[5-ethyl-2-(2-methoxy-phenyl)-4-(4-methoxy-thiazol-2-yl)-2H-pyrazol-3-ylamino]-5-methoxy-benzoic acid;
2-[4-(4-ethoxy-5-methyl-thiazol-2-yl)-5-ethyl-2-(2-methoxy-phenyl)-2H-pyrazol-3-ylamino]-5-methoxy-benzoic acid;
2-[5-ethyl-2-(2-methoxy-phenyl)-4-thiazol-2-yl-2H-pyrazol-3-ylamino]-5-methoxy-benzoic acid;
2-[5-ethyl-2-(2-methoxy-phenyl)-4-(4-methyl-thiazol-2-yl)-2H-pyrazol-3-ylamino]-5-methoxy-benzoic acid;
2-[5-ethyl-4-(4-ethyl-thiazol-2-yl)-2-(2-methoxy-phenyl)-2H-pyrazol-3-ylamino]-5-methoxy-benzoic acid;
2-[2-(2-chloro-phenyl)-4-(5-ethyl-4-methoxy-thiazol-2-yl)-5-methyl-2H-pyrazol-3-ylamino]-5-methoxy-benzoic acid;
2-[2-(2-chloro-phenyl)-4-(4-methoxy-5-methyl-thiazol-2-yl)-5-methyl-2H-pyrazol-3-ylamino]-5-methoxy-benzoic acid;
2-[4-(5-ethyl4-methoxy-thiazol-2-yl)-5-methyl-2-o-tolyl-2H-pyrazol-3-ylamino]-5-methoxy-benzoic acid;
2-[5-ethyl-4-(4-methoxy-thiazol-2-yl)-2-o-tolyl-2H-pyrazol-3-ylamino]-5-methoxy-benzoic acid;
2-[5-ethyl-4-(4-methoxy-5-methyl-thiazol-2-yl)-2-o-tolyl-2H-pyrazol-3-ylamino]-5-methoxy-benzoic acid;
2-(5-ethyl-4-thiazol-2-yl-2-o-tolyl-2H-pyrazol-3-ylamino)-5-methoxy-benzoic acid;
2-[5-ethyl-4-(4-methyl-thiazol-2-yl)-2-o-tolyl-2H-pyrazol-3-ylamino]-5-methoxy-benzoic acid;
2-[5-ethyl-4-(4-ethyl-thiazol-2-yl)-2-o-tolyl-2H-pyrazol-3-ylamino]-5-methoxy-benzoic acid;
2-[5-ethyl-2-(2-methoxy-phenyl-)4-(4-methoxy-thiazol-2-yl)-2H-pyrazol-3-ylamino]-5-methyl-benzoic acid;
2-[5-ethyl-4-(4-methoxy-5-methyl-thiazol-2-yl)-2-(2-methoxy-phenyl)-2H-pyrazol-3-ylamino]-5-methyl-benzoic acid;
5-methoxy-2-[2-(2-methoxy-phenyl)-4-(4-methoxy-thiazol-2-yl)-5-methyl-2H-pyrazol-3-ylamino]-benzoic acid;
5-methoxy-2-[4-(4-methoxy-5-methyl-thiazol-2-yl)-2-(2-methoxy-phenyl)-5-methyl-2H-pyrazol-3-ylamino]-benzoic acid;
2-[2-(2-methoxy-phenyl)-4-(4-methoxy-thiazol-2-yl)-5-methyl-2H-pyrazol-3-ylamino]-5-methyl-benzoic acid;
5-chloro-2-[2-(2-methoxy-phenyl)-4-(4-methoxy-thiazol-2-yl)-5-methyl-2H-pyrazol-3-ylamino]-benzoic acid;
5-chloro-2-[4-(4-methoxy-5-methyl-thiazol-2-yl)-2-(2-methoxy-phenyl)-5-methyl-2H-pyrazol-3-ylamino]-benzoic acid;
2-[4-(4-ethyl-thiazol-2-yl)-2-(2-methoxy-phenyl)-5-methyl-2H-pyrazol-3-ylamino]-5-methoxy-benzoic acid;
2-[4-(4-ethyl-thiazol-2-yl)-2-(2-methoxy-phenyl)-5-methyl-2H-pyrazol-3-ylamino]-5-methyl-benzoic acid;
2-[5-ethyl-2-(2-methoxy-phenyl)-4-(4-methoxy-thiazol-2-yl)-2H-pyrazol-3-ylamino]-benzoic acid;
5-ethyl-2-[5-ethyl-4-(4-methoxy-5-methyl-thiazol-2-yl)-2-(2-methoxy-phenyl)-2H-pyrazol-3-ylamino]-benzoic acid; and
5-ethyl-2-[5-ethyl-4-(4-ethyl-thiazol-2-yl)-2-(2-methoxy-phenyl)-2H-pyrazol-3-ylamino]-benzoic acid.

10. A pharmaceutical composition comprising an effective amount of a compound of claim 1, or a pharmaceutically acceptable salt thereof, in combination with a pharmaceutically acceptable carrier.

11. A pharmaceutical composition comprising a therapeutically effective amount of a compound of claim 1, or a pharmaceutically acceptable salt thereof, in combination with a pharmaceutically acceptable carrier and one or more pharmaceutical agents.

12. The pharmaceutical composition of claim 11, wherein said pharmaceutical agent is selected from the group consisting of PPAR ligands, insulin secretagogues, sulfonylurea drugs, α-glucosidase inhibitors, insulin sensitizers, hepatic glucose output lowering compounds, insulin and insulin derivatives, biguanides, protein tyrosine phosphatase-1B, dipeptidyl peptidase IV, 11beta-HSD inhibitors, anti-obesity drugs, HMG-CoA reductase inhibitors, nicotinic acid, lipid lowering drugs, ACAT inhibitors, bile acid sequestrants, bile acid reuptake inhibitors, microsomal triglyceride transport inhibitors, fibric acid derivatives, β-blockers, ACE inhibitors, calcium channel blockers, diuretics, renin inhibitors, AT-1 receptor antagonists, ET receptor antagonists, neutral endopeptidase inhibitors, vasopepsidase inhibitors, and nitrates.

13. A method of treating diabetes comprising the step of administering to a subject in need thereof a therapeutically effective amount of a compound of claim 1 or a pharmaceutical composition of claim 10.

14. The method of claim 13, wherein said diabetes is selected from the group consisting of type 1 diabetes, type 2 diabetes, maturity-onset diabetes of the young, latent autoimmune diabetes adult, and gestational diabetes.

15. A method of treating Syndrome X comprising the step of administering to a subject in need thereof a therapeutically effective amount of a compound of claim 1 or a pharmaceutical composition of claim 10.

16. A method of treating diabetes-related disorders comprising the step of administering to a subject in need thereof a therapeutically effective amount of a compound of claim 1 or a pharmaceutical composition of claim 10.

17. The method of claim 16, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

18. A method of treating or preventing secondary causes of diabetes comprising the step of administering to a subject in need thereof a therapeutically effective amount of a compound of claim 1 or a pharmaceutical composition of claim 10.

19. The method of claim 18, wherein said secondary cause is selected from the group consisting of glucocorticoid excess, growth hormone excess, pheochromocytoma, and drug-induced diabetes.

20. A method of treating diabetes comprising the step of administering to a subject in need thereof a therapeutically effective amount of a compound of claim 1 in combination with one or more pharmaceutical agents.

21. The method of claim 20, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents.

22. The method of claim 20, wherein said diabetes is selected from the group consisting of type 1 diabetes, type 2 diabetes, maturity-onset diabetes of the young, latent autoimmune diabetes adult, and gestational diabetes.

23. A method of treating Syndrome X comprising the step of administering to a subject in need thereof a therapeutically effective amount of a compound of claim 1 in combination with one or more pharmaceutical agents.

24. The method of claim 23, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents.

25. A method of treating diabetes-related disorders comprising the step of administering to a subject in need thereof a therapeutically effective amount of a compound of claim 1 in combination with one or more pharmaceutical agents.

26. The method of claim 25, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

27. The method of claim 26, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents.

28. A method of treating or preventing secondary causes of diabetes comprising the step of administering a subject in need thereof a therapeutically effective amount of a compound of claim 1 in combination with one or more pharmaceutical agents.

29. The method of claim 28, wherein said pharmaceutical agent is selected from the group consisting of PPAR agonists, sulfonylurea drugs, non-sulfonylurea secretagogues, α-glucosidase inhibitors, insulin sensitizers, insulin secretagogues, hepatic glucose output lowering compounds, insulin, and anti-obesity agents

30. A method of treating diabetes, Syndrome X, diabetes-related disorders or secondary causes of diabetes comprising the step of administering to a subject in need thereof a therapeutically effective amount of a compound of claim 1 in combination with one or more agents selected from the group consisting of HMG-CoA reductase inhibitors, nicotinic acid, lipid lowering drugs, ACAT inhibitors, bile acid sequestrants, bile acid reuptake inhibitors, microsomal triglyceride transport inhibitors, fibric acid derivatives, β-blockers, ACE inhibitors, calcium channel blockers, diuretics, renin inhibitors, AT-1 receptor antagonists, ET receptor antagonists, neutral endopeptidase inhibitors, vasopepsidase inhibitors, and nitrates.

31. The method of claim 31, wherein said diabetes-related disorder is selected from the group consisting of hyperglycemia, hyperinsulinemia, impaired glucose tolerance, impaired fasting glucose, dyslipidemia, hypertriglyceridemia, and insulin resistance.

32. The method of claim 20, wherein the compound of claim 1 and one or more pharmaceutical agents are administered as a single pharmaceutical dosage formulation.

33. A method of treating cardiovascular disease comprising the step of administering to a subject in need thereof a therapeutically effective amount of a polypeptide of claim 1 or a pharmaceutical composition of claim 10.

34. The method of claim 33, wherein said cardiovascular disease is selected from atherosclerosis, coronary heart disease, coronary artery disease, and hypertension.

35. A method of treating obesity comprising the step of administering to a subject in need thereof a therapeutically effective amount of a compound of claim 1 or a pharmaceutical composition of claim 10.

36. A method of stimulating insulin secretion in a subject in need thereof by administering to said subject a compound of claim 1 or a pharmaceutical composition of claim 10.

37. (canceled)

38. (canceled)

39. (canceled)

40. (canceled)
